(19) United States
(12) Patent Application Publication
(10) Pub. No.: US 2011/0142889 A1
(43) Pub. Date: Jun. 16, 2011

(54) COMPOSITIONS AND METHODS FOR ORAL DRUG DELIVERY

(75) Inventors: William W. Lee, San Diego, CA (US); Feng Lu, Shanghai (CN); Min Yin, Shanghai (CN)

(73) Assignee: NOD Pharmaceuticals, Inc., San Diego, CA (US)

(21) Appl. No.: 12/970,637

(22) Filed: Dec. 16, 2010

Related U.S. Application Data
(60) Provisional application No. 61/287,146, filed on Dec. 16, 2009.
(60) Provisional application No. 61/365,916, filed on Jul. 20, 2010.

(30) Foreign Application Priority Data
Dec. 16, 2009 (CN) 200910201248.3
Jul. 14, 2010 (CN) 201020227045.4
Dec. 16, 2010 (CN) 201010590909.9

(51) Int. Cl. A61K 9/00; A61K 38/00; A61K 31/715; A61K 31/7088; A61K 31/20; A61K 31/70; A61K 38/28; A61K 38/27; A61K 38/29; A61K 38/23; A61K 38/22; A61K 38/31; A61K 38/21; B05D 3/00; A61P 3/10; A61P 43/00; B29C 35/08

(52) U.S. Cl. 424/400; 514/1.1; 514/54; 514/44 R; 514/558; 514/23; 514/5.9; 514/7.7; 514/11.3; 514/6.7; 514/11.7; 514/11.8; 514/11.9; 514/9.7; 514/11.1; 424/85.4; 427/2.14; 264/400

(57) ABSTRACT

The invention provides a pharmaceutical composition for oral drug delivery comprising a solid dosage form containing an effective amount of a therapeutic agent, a permeation enhancer and a pharmaceutically acceptable excipient and a bioadhesive layer containing a bioadhesive polymer, and optionally comprising an impermeable or semi-permeable layer having an opening capable of directing a unidirectional release of the therapeutic agent and the permeation enhancer from the solid dosage form. Methods of making and using the present pharmaceutical composition are also provided.

### CROSS-REFERENCE TO RELATED APPLICATIONS

This application claims the benefit of U.S. Ser. Nos. 61/287,146, filed Dec. 16, 2009, and 61/365,916, filed Jul. 20, 2010, which are incorporated herein by reference in their entireties.

### FIELD OF THE INVENTION

The present invention generally relates to the field of oral drug delivery, and in particular to pharmaceutical compositions for enhancing absorption and increasing bioavailability of therapeutic agents that demonstrate poor absorption and low bioavailability in conventional oral drug delivery systems. The invention further relates to methods for making and using the disclosed pharmaceutical compositions.

### BACKGROUND OF THE INVENTION

Oral drug delivery is one of the most common and accepted routes of drug administration. However, many therapeutic agents are poorly delivered via the oral route. For example, biologically active macromolecules such as proteins, peptides, polysaccharides and nucleic acids often cannot be administered orally due to the combined effects of enzyme degradation, poor absorption or instability. Similarly, oral formulations of many classes of small molecule drugs such as cyclosporine, fenofibrate, lipid lowering statins, antihypertensive sartans, antibiotics like ceftriaxone or azithromycin, and bisphosphonate clodronrate suffer from poor absorption and variable pharmacokinetic profiles.

A number of different approaches have been explored to improve oral delivery of poorly absorbed therapeutic agents. For example, permeation enhancers are commonly used to enhance the absorption of drugs that are otherwise poorly absorbed (for a review, see B. J. Aungst, J. Pharm. Sci., 2000, 89(4):429-442). Numerous examples of permeation enhancers that are known to improve transdermal or transmucosal absorption are disclosed in U.S. Pat. Nos. 4,525,339, 4,722,941, 5,318,781, 5,393,738, 5,424,289, 5,597,562, 5,714,477, 5,817,624, 5,827,534, 5,854,281, 5,912,014, 5,929,027, 5,952,000, 5,972,911, 6,071,538, 6,156,731, 6,200,602, 6,333,046, 6,423,334, 6,747,014, 7,316,819, 7,576,067; U.S. Patent Application Pub. Nos. 2007/0148228, 2007/0196464, 2007/0238707, 2008/0275001, 2008/0299079, 2009/0087484, 2009/0111736 and European Patent EP 1 154 761.

The application of absorption enhancers in oral formulations is often limited due to associated toxicity. One documented example of a successful pharmaceutical product containing an absorption enhancer is a colon suppository formulation of ampicillin, which is commercially available in Sweden (DOKTACILLIN™, Astra Lakemedel AB). A formulation containing 25 mg sodium caprate as a permeation enhancer was reported to increase the maximum serum concentration (Cₘₐₓ), area under the serum concentration-time curve (AUC) and urinary recovery of ampicillin 2.6-, 2.3- and 1.8-fold, respectively, compared to ampicillin alone (T. Lindmark et al., Pharm. Res., 1997, 14(7):930-935).

However, the colonic environment is quite different from the oral route in terms of motility, residence time, water flow, mucus and intestinal content. Therefore, the amount of permeation enhancer required for oral delivery is considerably higher. For example, Burcham et al. studied the effects of sodium caprate and other permeation enhancers on the absorption of the peptide mimic drug DMP 728 in dogs (Pharm. Res., 1995, 12(12):2065-7200). While a formulation containing 115-120 mg sodium caprate in gelatin capsules produced a modest improvement in bioavailability (from 13.0% to 17.7%), an enteric formulation containing the same amount of sodium caprate failed to show any effect.

To obtain consistent and significant enhancement in absorption and bioavailability, the amount of sodium caprate required in the same experimental system was found to be in the range of 275-550 mg per tablet (U.S. Patent Application Pub. No. 2008/0275001). However, the high amount of permeation enhancer required for oral drug delivery often causes toxicity and triggers safety concerns.

To date, no safe and effective solution has been found for the problem of oral delivery of therapeutic agents having poor absorption and bioavailability, particularly for macromolecular biopharmaceutical products. Accordingly, there is a need to develop a drug delivery system that can produce a significant improvement in absorption and bioavailability without using an excessive amount of a permeation enhancer.

### SUMMARY OF THE INVENTION

One object of the present invention is to provide a non-toxic pharmaceutical composition capable of enhancing the absorption and/or bioavailability of a poorly absorbed therapeutic agent. Another object is to provide a pharmaceutical composition for enhanced drug delivery that can modulate the pharmacokinetic profile of a therapeutic agent and is inexpensive and relatively easy to manufacture.

In one embodiment, the invention provides a pharmaceutical composition for drug delivery comprising a solid dosage form containing an effective amount of a therapeutic agent, a permeation enhancer and a pharmaceutically acceptable excipient, to form a core, which is coated by a bioadhesive layer containing a bioadhesive polymer.

In some embodiments, the above pharmaceutical composition can be further coated with an enteric material to prevent content release in the stomach and permit release at a preferred site in the gastrointestinal tract such as the small intestine.

The inventors surprisingly found that the presence of a bioadhesive polymer layer allows a reduction in the amount of a permeation enhancer required to significantly improve absorption and bioavailability of a poorly absorbed therapeutic agent.

In some embodiments, the invention provides a pharmaceutical composition for drug delivery comprising a solid dosage form containing an effective amount of a therapeutic agent, a permeation enhancer and a pharmaceutically acceptable excipient to form a core, which is coated by a bioadhesive layer containing a bioadhesive polymer, and further coated by an impermeable or semi-permeable layer having an opening capable of directing a substantially unidirectional release of the therapeutic agent and the permeation enhancer from the solid dosage form.

In some embodiments, the pharmaceutical composition can be further coated with an enteric material to prevent content release in the stomach and permit release at a preferred site in the gastrointestinal tract such as the small intestine.

In some embodiments, the therapeutic agent and the permeation enhancer have substantially equivalent relative rates of release from the solid dosage form. Thus, the present pharmaceutical composition allows regional, restricted and substantially synchronous release of a therapeutic agent and a permeation enhancer, thereby improving the absorption of the therapeutic agent using a significantly reduced amount of the permeation enhancer compared to the prior art.

In another aspect, the invention provides a pharmaceutical composition that is capable of modulating the pharmacokinetic profile of a therapeutic agent. The release kinetics of the therapeutic agent and the permeation enhancer can be modulated by a different composition and/or ratio of components in the core matrix, or by a different composition, ratio, and/or thickness of the bioadhesive polymer layer, or by a different composition, ratio, and/or thickness of the impermeable or semi-permeable layer, to provide a burst or immediate release, or an extended or sustained profile, depending on the desired therapeutic effect.

In yet another aspect, the invention provides a method for making a pharmaceutical composition for drug delivery comprising fabricating a solid dosage form comprising an effective amount of a therapeutic agent, a permeation enhancer and a pharmaceutically acceptable excipient; coating the solid dosage form with a bioadhesive layer comprising a bioadhesive polymer; and optionally coating the solid dosage form with an impermeable or semi-permeable layer comprising an opening capable of directing a substantially unidirectional release of the therapeutic agent and the permeation enhancer from the solid dosage form.

In some embodiments, the order of applying the bioadhesive polymer layer and the impermeable or semi-permeable layer is reversed. In some embodiments, the method further comprises coating the composition with an enteric layer.

In yet another aspect, the invention provides a method of treating a subject in need of a therapeutic treatment by administering the present pharmaceutical composition to the subject by the oral, nasal, buccal, sublingual, rectal or vaginal routes of administration.

### BRIEF DESCRIPTION OF THE DRAWINGS

FIG. 1 illustrates the synchronous release of exenatide and absorption enhancer sodium caprate from tablets coated with layers of hydroxypropyl methylcellulose (HPMC) and enteric polymer EUDRAGIT® L30D-55.

FIGS. 2A and 2B show the effect of a unidirectional release layer on the release kinetics of exenatide and sodium caprate from tablets coated with layers of HPMC and enteric polymer.

FIGS. 3A and 3B show the effect of a bioadhesive layer on the bioavailability of exenatide in the presence of 100 and 400 mg sodium caprate and an enteric layer.

FIG. 4 demonstrates the effect of sodium caprate amount on the bioavailability of exenatide in the presence of an AA1 layer and an enteric layer.

FIG. 5 shows the effect of a unidirectional release layer on the bioavailability of exenatide in the presence of 50 mg caprate, an HPMC layer, and an enteric layer.

FIG. 6 shows the effect of a unidirectional release layer on the bioavailability of exenatide in the presence of 100 mg caprate, an HPMC layer, and an enteric layer.

FIG. 7 shows the effect of an enteric layer on the bioavailability of exenatide in the presence of 200 mg sodium caprate, an HPMC layer, and a unidirectional release layer.

FIG. 8A illustrates the release kinetics of insulin in 0.01N HCl (first 2 hrs) and simulated intestinal fluid (SIF), pH 6.8 (hours 3-7) in the presence of 200 mg sodium caprate, an HPMC layer, and a unidirectional release layer. FIGS. 8B and 8C demonstrate the effects of oral insulin treatment on blood glucose and serum insulin concentration, respectively, in somatostatin treated dogs.

FIG. 9 compares the effects of two oral insulin formulations on blood glucose levels in somatostatin treated dogs. The first formulation was a two-layer tablet that included 200 mg sodium caprate, an HPMC layer and a unidirectional release layer. The second formulation was a three-layer tablet that included 200 mg sodium caprate, an HPMC layer, a unidirectional release layer, and an enteric outer layer.

FIGS. 10A-C compare stability of exenatide in various solid formulations following storage at 60° C. (FIG. 10A), 25° C. at 92.5% relative humidity (FIG. 10B), and 4500 lux photo exposure (FIG. 10C) for 0, 5 and 10 days.

FIG. 11 shows the effect of oral insulin administration on blood glucose in normal dogs. The insulin formulation used for this experiment was a three-layer tablet that included 100 sodium caprate, an HPMC layer, a unidirectional release layer, and an enteric layer.

FIG. 12 compares exenatide absorption mediated by unidirectional release layers produced by manual and laser ablation processes. The exenatide formulations used for this experiment were two-layer tablets that included 200 mg sodium caprate, an HPMC layer containing the L30D-55 enteric material, and a unidirectional release layer prepared either manually or by laser ablation.

FIG. 13 illustrates the dose-dependent effect of oral insulin administration (25U, 25U×2, and 50U) on blood glucose levels in somatostatin treated dogs. The insulin formulations used for this experiment were two-layer tablets that included 200 mg sodium caprate, an HPMC layer containing the L30D-55 enteric material, and a unidirectional release layer.

FIGS. 14A and 14B demonstrate the effect of bioadhesive polymer content in the bioadhesive layer on the kinetics of exenatide release and absorption in normal dogs. The exenatide formulations used for this experiment were two-layer tablets that included 100 mg sodium caprate, an HPMC layer containing 65% or 80% HPMC and the L30D-55 enteric material, and a unidirectional release layer prepared by laser ablation.

### DETAILED DESCRIPTION OF THE INVENTION

### Terms and Definitions

Unless defined otherwise, all technical and scientific terms used herein have the same meaning as is commonly understood by one of ordinary skill in the art to which this invention belongs. All patents, published patent applications, and other publications and databases referred to herein are incorporated by reference in their entireties. If a definition set forth in this section is contrary to or otherwise inconsistent with a definition set forth in patents, published patent applications and other publications and other data bases that are herein incorporated by reference, the definition set forth in this section prevails over the definition that is incorporated herein by reference.

Citation of publications or documents is not intended as an admission that any of such publications or documents are pertinent prior art, nor does it constitute any admission as to the contents or date of these publications or documents.

As used herein, “a” or “an” means “at least one” or “one or more.”

As used herein, the term “pharmaceutical composition” or “pharmaceutically acceptable formulation” refers to a composition or formulation that allows for the effective distribution of a moiety or a compound in the physical location most suitable for its desired activity.

As used herein, the term “effective amount” or “therapeutically effective amount” of an active agent refers to a nontoxic but sufficient amount of the agent to provide the desired therapeutic or prophylactic effect to most patients or individuals. It is commonly recognized that the effective amount of a pharmacologically active agent may vary depending on the route of administration, as well as the age, weight, and sex of the individual to which the drug or pharmacologically active agent is administered. It is also commonly recognized that one of skill in the art can determine appropriate effective amounts by taking into account such factors as metabolism, bioavailability, and other factors that affect plasma levels of an active agent following administration within the unit dose ranges disclosed further herein for different routes of administration.

As used herein, the term “pharmaceutically acceptable” refers to a non-toxic, inert composition that is physiologically compatible with humans or other mammals.

As used herein, the term “pharmaceutical excipient” refers to a material such as an adjuvant, a carrier, pH-adjusting and a buffering agent, a tonicity adjusting agent, a wetting agent, a preservative, and the like.

As used herein, the terms “subject,” “individual,” “host,” and “patient” are used interchangeably to refer to an animal that is the object of treatment, observation and/or experiment. The term “animal” includes vertebrates and invertebrates, such as fish, shellfish, reptiles, birds, and, in particular, mammals. The term “mammal” encompasses, without limitation, mice, rats, rabbits, guinea pigs, dogs, cats, sheep, goats, cows, horses, primates, such as monkeys, chimpanzees and apes, and, in particular, humans.

As used herein, the term “treating” refers to any and all uses which remedy or prevent a diseased or infected state or symptoms, or otherwise deter, hinder, retard, or reverse the progression of a disease/infection or other undesirable symptoms. As used herein, the terms “treating” and “therapeutic” refer to any improvement or amelioration of any consequence of disease; full eradication of disease is not required. Amelioration of symptoms of a particular disorder refers to any lessening of symptoms, whether permanent or temporary, that can be attributed to or associated with administration of a therapeutic composition of the present invention.

As used herein, the terms “administration” or “administering” refers to any suitable method of providing a composition of the present invention of the invention to a subject. The pharmaceutical compositions of the present invention may be administered by the oral, nasal, buccal, sublingual, rectal or vaginal routes of administration. The pharmaceutical compositions may be formulated in suitable dosage unit formulations appropriate for each route of administration.

As used herein, the term “solid dosage form” refers to any dosage form that is in the form of a solid including, but not limited to, tablets, caplets, capsules including those made from hard or soft materials such as gelatin or natural or synthetic gelatin substitutes, lozenges, combinations thereof and the like.

As used herein, the term “permeation enhancer” refers to an agent that improves the rate of transport of a pharmacologically active agent across the mucosal surface. Typically, a permeation enhancer increases the permeability of mucosal tissue to a therapeutic agent. For example, permeation enhancers increase the rate at which the therapeutic agent permeates through membranes and enters the bloodstream. Enhanced permeation effected through the use of permeation enhancers can be observed, for example, by measuring the flux of the pharmacologically active agent across animal or human membranes. As used herein, an “effective” amount of a permeation enhancer refers to an amount that will provide a desired increase in mucosal membranes permeability to provide, for example, the desired absorption and/or bioavailability of a selected compound.

As used herein, the term “bioadhesive” generally refers to any adhesive that interfaces with living tissue and/or biological fluid. The term “bioadhesive layer” refers to a solid layer that is intended to be adhered to a mucosal tissue of a subject. The bioadhesive layer contains at least one “bioadhesive polymer,” which may be selected, without limitation, from carbomer, polycarbophil, hydroxypropyl methylcellulose, hydroxyethyl cellulose, hydroxypropyl cellulose, carboxymethyl cellulose, polyvinyl alcohol, sodium hyaluronate, chitosan, alginate, xanthum gum, acrylic polymers and derivatives and mixtures thereof.

As used herein, the term “impermeable or semi-permeable” refers to a material that is sufficiently impermeable to physiological fluids as well as ingredients contained within the drug delivery system such that the migration of such fluids and ingredients into or out of the system through the impermeable or semi-permeable material is so low as to have substantially no adverse impact on the function of the system.

As used herein, the term “substantially unidirectional release” means that more than about 50%, 60%, 70%, preferably more than about 80%, more preferably more than about 90% and most preferably more than about 95% of the therapeutic agent and the permeation enhancer comprised in the solid dosage form is released from the solid dosage form in the same single direction that is defined by an opening in the impermeable or semi-permeable layer.

As used herein, the term “substantially equivalent relative rates of release” means that the release of the therapeutic agent and the permeation enhancer from the solid dosage form is substantially synchronous, i.e. the difference between the fraction (%) of the therapeutic agent released at any given time and the fraction of the permeation enhancer released at the same time is less than about 50%, 40%, 30%, preferably less than about 20%, more preferably less than about 10% and most preferably less than about 5%.

As used herein, the terms “enteric coating,” “enteric layer,” “enteric material” and “enteric polymer” refer to a mixture of pharmaceutically acceptable excipients which is applied to the solid dosage form and which prevents release of the active ingredient(s) in the mouth, esophagus or stomach, but which rapidly and completely releases the drug when the dosage form passes into the proximal portion of the lower gastrointestinal tract. The enteric layer preferably comprises about 1 to 15%, more preferably about 3 to 12%, and most preferably about 6 to 10% by weight based on the combined weight of the solid dosage form and the coating. The enteric coating polymer may be selected, without limitation, from cellulose acetate phthalate (EUDRAGIT® S or L), hydroxypropyl methylcellulose phthalate, hydroxypropyl methylcellulose acetate succinate, cellulose acetate trimellitate, polyvinyl acetate phthalate, shellac and methacrylic acid copolymers. The thickness of the coating is selected to provide the desired release rate, which is dependent on the nature and thickness of the coating.

As used herein, the term “plasticizer” refers to a material that may be incorporated into the pharmaceutical composition in order to decrease the glass transition temperature and the melt viscosity of a polymer by increasing the free volume between polymer chains. Plasticizers include, but are not limited to, citrate esters (e.g., triethylcitrate, triacetin), low molecular weight polyalkylene oxides (e.g., polyethylene glycols, polypropylene glycols, polyethylene/propylene glycols), glycerol, pentaerythritol, glycerol monoacetate, diacetate or triacetate, propylene glycol, and sodium diethyl sulfosuccinate. The plasticizer can be present in a concentration from about 0.1% to about 25%, preferably about 0.5-15% or about 1-20% by weight of the pharmaceutical composition. Additional examples of plasticizers can be found in M. & I. Ash, THE HANDBOOK OF PHARMACEUTICAL ADDITIVES (3^{rd} ed., Synapse Information Resources, Inc., 2007).

Throughout this disclosure, various aspects of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 3, from 2 to 4, from 2 to 5, from 2 to 6, etc., as well as individual numbers within that range, for example, 1, 2, 3, 4, 5 and 6. This applies regardless of the breadth of the range.

### Drug Delivery Systems

As noted above, the invention provides a pharmaceutical composition for drug delivery comprising a solid dosage in the form of tablet, patch, disc or powder comprising an effective amount of a therapeutic agent, a permeation enhancer and a pharmaceutically acceptable excipient to form a core, which is further coated with a bioadhesive layer containing a bioadhesive polymer.

In some embodiments, the pharmaceutical composition is further coated with an enteric material to permit release at a preferred site in the gastrointestinal tract.

Although membrane coating with polymers such as hydroxypropylmethyl cellulose, polyvinyl alcohol, and polyethylene glycol is commonly practiced to enhance the enteric coating process, the inventors surprisingly found that the presence of a bioadhesive polymer layer allows reducing the amount of a permeation enhancer required to significantly improve absorption and bioavailability of a poorly absorbed therapeutic agent.

Examples of the disclosed pharmaceutical composition are illustrated in the working examples. In the examples, the permeation enhancer was sodium caprate and the drug tested was exenatide, which is a 39-amino acid peptide (marketed by Amylin and Eli Lilly as BYETTA® for the treatment of type 2 diabetes). In the prior art, the amount of sodium caprate needed to achieve a significant absorption enhancement was in the range of 275-550 mg in dogs (U.S. Patent Application Pub. No. 2008/0275001). A smaller amount of sodium caprate (115-120 mg) in an enteric coated formulation was found to be ineffective in the same animal model (Burcham et al., Pharm. Res., 1995, 12(12):2065-2070).

The inventors found that, consistent with the prior art, a formulation containing 100 mg sodium caprate alone was also ineffective. Surprisingly, however, formulations containing the same amount of sodium caprate (100 mg) and a bioadhesive layer were equally or more effective compared with those formulations containing a higher amount of sodium caprate alone. When the bioadhesive layer was incorporated in formulations containing a higher amount sodium caprate (400 mg), a further enhancement of absorption was observed. It was completely unexpected that the presence of a bioadhesive layer would affect the requirement for a permeation enhancer to such an appreciable extent.

In some embodiments, the invention provides a pharmaceutical composition for drug delivery comprising a solid dosage in the form of tablet, patch, disc or powder containing an effective amount of a therapeutic agent, a permeation enhancer and a pharmaceutically acceptable excipient to form a core, which is further coated with a bioadhesive layer containing a bioadhesive polymer, which is then further coated with an impermeable or semi-permeable layer having an opening capable of directing a substantially unidirectional release of the therapeutic agent and the permeation enhancer from the solid dosage form.

In some embodiments, the pharmaceutical composition can be further coated with an enteric material to permit release at a preferred site in the gastrointestinal tract. Surprisingly, it was found that this composition can further reduce the amount of a permeation enhancer without adversely affecting the absorption enhancement.

The prior art contains different examples of unidirectional release dosage forms. For instance, U.S. Pat. Nos. 4,772,470 and 5,827,525 disclose a patch or oral bandage for buccal drug administration. Similarly, an oral patch format comprising a site selecting layer, a drug, an adhesive layer and impermeable layer is disclosed in U.S. Pat. No. 7,097,851 and a number of non-patent publications (e.g., S. Eaimtrakarn et al., Biomaterials, 2002, 23(1):145-152; S. Eaimtrakarn et al., Intl. J. Pharm., 2003, 250(1):111-117; and S. L. Tao & T. A. Desai, Drug Discov. Today, 2005, 10(13):909-915). A bioadhesive patch coated with an impermeable ethyl cellulose layer was reported to deliver insulin in a rat in situ model, and permeation enhancers were found not to be critical for this delivery system to work (K. Whitehead et al., J. Control. Release, 2004, 98(1):37-45). All of these publications are incorporated herein by reference in their entireties.

Notably, even though a number of patents and non-patent publications have disclosed the oral patch format and unidirectional release dosage forms, there have been no subsequent commercial applications reported in this pharmaceutically important field, indicating that the oral patch format continues to pose significant technical challenges.

The effect of the unidirectional release layer on the absorption and bioavailability of exenatide in the presence of sodium caprate is illustrated in Example 6. In a formulation containing 50 mg sodium caprate and no unidirectional release layer, exenatide absorption in dogs was minimal, consistent with the earlier reports in the art (U.S. Pat. No. 7,605,123). However, when a unidirectional release layer was applied, exenatide absorption was significantly enhanced. The coating material can be either impermeable or semi-permeable.

It is known in the art that permeation enhancers often require a certain minimal concentration to be effective. For example, the concentration of sodium caprate needed to achieve a permeation enhancing effect is estimated to be at least 10-13 mM (see E. K. Anderberg et al., Pharm. Res., 1993, 10(6):857-864). It is further recognized that the release of the permeation enhancer needs to be relatively fast and substantially synchronous with that of the therapeutic agent to avoid rapid dilution in the gastrointestinal tract, which is under constant fluid flow. For instance, U.S. Patent Application Pub. No. 2008/0275001 discloses immediate release (IR) and sustained release (SR) formulations of low molecular weight heparin containing sodium caprate as a permeation enhancer. Given the same amount of sodium caprate, the sustained release formulation was significantly less effective compared to the immediate release formulation.

Accordingly, it was surprising to find that formulations comprising an additional impermeable or semi-permeable layer and having an extended release profile compared to simpler formulations have demonstrated a relatively high level of absorption enhancement. The inventors have further found that unidirectional release formulations may be used to modulate a therapeutic agent's pharmacokinetic profile by varying the thickness of the impermeable or semi-permeable layer, the content and nature of the plasticizer used to modify the impermeable or semi-permeable material, and the size of the opening. In some embodiments, the therapeutic agent and the permeation enhancer are released from the pharmaceutical composition in a substantially synchronous fashion. In some embodiments, the pharmaceutical composition extends the time of release and allows sustained absorption.

### Therapeutic Agents

In some embodiments, the therapeutic agents to be delivered comprise those that are poorly absorbed in the gastrointestinal tract and belong to class III or IV compounds according to the Biopharmaceutics Classification System (BCS) classification (see Food and Drug Administration, “Guidance for Industry: Waiver of In Vivo Bioavailability and Bioequivalence Studies for Immediate-Release Solid Oral Dosage Forms Based on a Biopharmaceutics Classification System”).

In some embodiments, the poorly absorbed therapeutic agent is selected from the group consisting of an acetylcysteine, an acamprosate, an acyclovir, an albendazole, an alcuronium, an alendronate, an alfuzosin, an alprazolam, an alprostadil, an amikacin, an aminobisphosphonate, an amiodarone, an amitriptyline, an amlodipine, an amoxacilline, an amphetamine, an amphotericin B, an ampicillin, an artemether, an artesunate, an aspirin, an atazanavir, an atenolol, an atomoxetine, an atorvastatin, an atropine, an azithromycin, an AZT, a bacitracin, a beclometasone, a benzathine benzylpenicillin, a benzylpenicillin, a biperiden, a bleomycin, a bosentan, a bupivacaine, a buprenorphine, a bupropion, a candesartan, a candoxatril a capreomycin, a captopril, a carbamazepine, a carbidopa, a carvedilol, caspofungin, a cefazolin, a cefdinir, a cefixime, a cefotaxime, a ceftazidime, a ceftriaxone, a celecoxib, a chlorambucil, a chloramphenicol, a chloroquine, a chlorphenamine, a chlorpromazine, a cilastatin, a cimitidine, a ciprofloxacin, a clarithromycin, a clofazimine, a clomipramine, a clonidine, a clopidogrel, a clotrimazole, a cloxacillin, a cyclophosphamide, a cyclosporine, a cytarabine, a d-9-tetrahydro cannabinol, a dacarbazine, a dactinomycin, a danazol, a dapsone, a daunorubicin, a deferoxamine, a desipramine, a dexamethasone, a didanosine, a diethylcarbamazine, a digoxin, a dihydroergotamine, a diltiazem, a dimercaprol, a dolargin, a domperidone, a domperidone, a dopamine, a doxazosin, a doxetaxel, a doxorubicin, a duloxetine, an efavirenz, an eflornithine, an enalapril, an enprostil, an epinephrine, an ergometrine, an erlotinib, an erythromycin, an esomeprazole, an estradiol, an eszopiclone, an etoposide, an ezetimibe, a famotidine, a felodipine, a fenofibrate, a fentanyl, a fexofenadine, a finasteride, a flucytosine, a fludrocortisones, a fluorouracil, a fluoxetine, a fluphenazine, a flurbiprofen, a fluticasone, a fluvastatin, a formoterol, a furosemide, a gabapentin, a ganciclovir, a gemcitabine, a gentamicin, a glibenclamide, a glimepiride, a glyceryl trinitrate, a griseofulvin, a griseofulvin, a haloperidol, a hydralazine, a hydrochlorothiazide, a hydrocortizone, a hydroxocobalamin, an ibandronic acid, an ibuprofen, an imipenem, an imipramine, an indinavir, an ipratropium bromide, an irbesartan, an irinotecan, an isoniazid, an isosorbide dinitrate, an itraconazole, a kanamycin, a ketoconazole, a ketoprofen, a labetalol, a latanoprost, a levamisole, a levodopa, a lidocaine, a lisinopril, a loperamide, a lopinavir, a losartan, a lovastatin, a lumefantrine, a mebendazole, a medroxyprogesterone, a mefloquine, a meglumine antimoniate, a melarsoprol, a mercaptopurine, a mesna, a metformin, a methadone, a methotrexate, a methyldopa, a methylphenidate, a methylthioninium chloride, a metoprolol, a mifepristone, a misoprostol, a modafinil, a mometasone, a montelukast, a morphine, a nadolol, a naloxone, a naproxen, a neostigmine, a nevirapine, a niclosamide, a nifedipine, a nifurtimox, a nitrofurantoin, a norethisterone, a nortriptylene, a nystatin, an ofloxacin, an olmesartan, an omeprazol, an ondansetron, an oxaliplatin, a paclitaxel, a pamidronate, a p-aminosalicylic acid, a paromomycin, a pemetrexed, a penicillamine, a pentamidine, a phenoxymethylpenicillin, a phenylacetic acid mustard, a phenyloin, a phytomenadione, a phytosterol, a piroxcam, a pilocarpine, a piperacillin, a pravastatin, a praziquantel, a prazosin, a prednisolone, a prednisone, a procaine benzylpenicillin, a procarbazine, a progesterone, a proguanil, a promethazine, a propranolol, a propanol, a propylthiouracil, a prostaglandin, a pyrantel, a pyridostigmine, a quetiapine, a quinidine, a quinine, a rabeprazole, a raloxifene, a ramipril, a ranitidine, a rapamycin, a ribavirin, a risedronic acid, a ritonavir, a ropinirole, a rosuvastatin, a salbutamol, a salicylic acid, a salmeterol, a saquinavir, a scopolamine, a sertraline, a sildenafil, a simvastatin, a sodium nitroprusside, a spectinomycin, a stavudine, a steroid, a stibogluconate, a stigmasterol, a sulfadoxine, a sulfamethoxazole, a sulfasalazine, a sumatriptan, a suramin, a suxamethonium, a tacrolimus, a tadalafil, a tamoxifen, a tegaserod, a telmisartan, a temozolomide, a tenidap, a tenofovir, a tenofovir disoproxil fumarate, a terfenadine, a testosterone, a tetracaine, a tetracycline, a timolol, a tiotropium, a triamcinalone, a triclabendazole, a trovafloxacin, a tubocurarine, a ubiquinone, a valaciclovir, a valproic, a valsartan, a vancomycin, a vardenafil, a vecuronium, a venlafaxine, a verapamil, a vinblastine, a vincristine, a vitamin B12, a zidovudine, a ziprasidone, a zoledronic acid, a zolpidem, its salts, analogs and derivatives.

In some embodiments, the therapeutic agent comprises one that is selectively absorbed at a specific absorption site including, without limitation, the upper small intestine. The therapeutic agents include, without limitation, riboflavin, levodopa, metformin, and furosemide.

In some embodiments, the therapeutic agent comprises a biologically active macromolecule selected from a protein, a peptide, a polysaccharide, a nucleic acid, a lipid, a carbohydrate or a combination thereof.

In some embodiments, the protein is selected from the group consisting of an anti-thrombin, an albumin, an alpha-1-proteinase inhibitor, an anti-hemophilic factor, a coagulation factor, an antibody, an anti-CD20 antibody, an anti-CD52 antibody, an anti-CD33 immunotoxin, a DNase, an erythropoietin, a factor IX, a factor VII, a factor VIII, a follicle stimulating hormone, a granulocyte colony-stimulating factor (G-CSF), a pegylated G-CSF, a galactosidase alpha or beta, a glucagon, a glucocerebrosidase, a granulocyte-macrophage colony-stimulating factor (GM-CSF), a choriogonadotropin, a hepatitis B antigen, a hepatitis B surface antigen, a hepatitis B core antigen, a hepatitis B envelopment antigen, a hepatitis C antigen, a hirudin, an anti-HER-2 antibody, an anti-IgE antibody, an anti-IL-2 receptor antibody, an insulin, an insulin glargine, an insulin aspart, an insulin detemir, an insulin lispro, an interferon, a pegylated interferon, an interferon alpha or alpha 2a or alpha 2b or consensus, an interferon beta or beta-1a or beta-1b or betaser, an interferon gamma, an interleukin-2, an interleukin-11, an interleukin-12, a luteinizing hormone, a nesiritide, an osteogenic protein-1, an osteogeneic protein-2, a lyme vaccine, a platelet derived growth factor, an anti-platelet antibody, an anti-RSV antibody, a somatotropin, an anti-tumor necrosis factor (TNF) antibody, a TNF receptor-Fc fusion protein, a tissue plasminogen activator (tPA), a TNK-tPA, a thyroid stimulating hormone (TSH), a fibrinolytic enzyme, a thrombolytic enzyme, an adenosine deaminase, a pegylated adenosine deaminase, an anistreplase, an asparaginase, a collagenase, a streptokinase, a sucrase, a urokinase, an aprotinin, a botulinum toxin, a fibroblast growth factor, a vascular endothelia growth factor, a venom, an antibody, an antibody fragment, and any combination thereof. The proteins may be produced by recombinant technology, chemical synthesis or extracted from biological sources. The proteins also include modified analogs or derivatives of wild type. The origin of the proteins may be human or from other species.

In some embodiments, the peptide is selected from the group consisting of an ACTH, an anti-angiogenic peptide, an adamtsostatin, an adiponectin, an adipokinetic hormone, an deiponutrin, an adipose desnutrin, an adrenomedullin, an agouti-related protein, an alarin, an allatostatin, an amelogenin, a calcitonin, an amylin, an amyloid, an agiopoietin, an angiotensin, an anorexigenic peptide, an anti-inflammatory peptide, an anti-diuretic factor, an anti-microbial peptide, an apelin, an apidaecin, a RGD peptide, an atrial natriuretic peptide, an atriopeptin, an auriculin, an autotaxin, a bombesin, a bombinakinin, a bradykinin, a brain natriuretic peptide, a brain-derived neutrophic factor, a brevinin, a C-peptide, a caspase inhibitor, a pancreatic peptide, a buccalin, a bursin, a C-type natriuretic peptide, a calcitonin related peptide, a calcitonin receptor stimulating peptide, a calmodulin, a CART, a cartilostatin, a casomokinin, a casomorphin, a catestatin, a cathepsin, a cecropin, a cerebellin, a chemerin, a chelocystokinin, a chromogranin, a ciliary neutrophic factor, a conantokin, a conopressin, a conotoxin, a copeptin, a cortical androgen stimulating hormone, a corticotropin release factor, a cortistatin, a coupling factor, a defensin, a delta sleep inducing peptide, a dermorphin, a vasopressin, a desamino-vasopressin, a diuretic hormone, a dynorphin, an endokinin, an endomorphin, an endorphin, an endostatin, an endothelin, an enkephalin, an enterostatin, an exendin, an exendin-4, an erythropoietic peptide, an epithelia growth factor, a fat targeted peptide, a galanin, a gastric inhibitory peptide, a gastrin, a gastrin releasing peptide, a ghrelin, a glucagon, a glucagon-like peptide, a glutathione derivative, a gluten exorphin, a growth hormone releasing factor, a GM-CSF inhibitory peptide, a growth hormone peptide, a guanylin, a HIV peptide, a helodemine, a hemokinin, a HCV peptide, a HBV peptide, a HSV peptide, a Herpes virus peptide, a hirudin, a hydra peptide, an insulin-like growth factor, a hydrin, an intermedin, a kassinin, a keratinocyte growth factor, a kinetensin, a kininogen, a kisspeptin, a kyotorphin, a laminin peptide, a leptin peptide, a leucokinin, a leucopyrokinin, a leupeptin, a luteinizing hormone releasing hormone (LHRH), a lymphokine, a melanin concentrating hormone and its inhibitor, a melanocyte stimulating hormone releasing inhibitor, a melanotropin-potentiating factor, a morphine modulating neuropeptide, a MSH, a neoendorphin, a nesfatin, a neurokinin, a neuromedin, a neutropeptide Y, a neurotensin, a neutrotrophic factor, a nociceptin, an obestatin, an opioid receptor antagonist, an orexin, an osteocalcin, an oxytocin, a pancreastatin, a peptide YY, a physalaemin-like peptide, a secretin, a somatostatin, a sperm-activating peptide, a substance P, a syndyphalin, a thrombospondin, a thymopoietin, a thymosin, a thyrotropin-releasing hormone, a transforming growth factor, a tuftsin, a tumor necrosis factor antagonist or related peptide, a usrechistachykinin, a urocortin, a urotensin antagonist, a valorphin, a vasotocin, a VIP, a xenopsin or related peptide, and any combination thereof. The peptides may be produced by recombinant technology, chemical synthesis or extracted from biological sources. The peptides include modified analogs or derivatives of wild type proteins. The origin of the peptides may be human or from other species.

In some embodiments, the biologically active macromolecule is a vaccine against a microorganism selected from a group consisting of an adenovirus, anthrax, BCG, botulinum, cholera, diphtheria toxoid, diphtheria & tetanus toxoids, diphtheria tetanus & pertussis, haemophilus B, hepatitis A, hepatitis B, influenza, encephalitis, measles, mumps, rubella, meningococcal, plague, pertussis, pneumococcal, polio, rabies, rotavirus, rubella, smallpox, tetanus toxoid, typhoid, varicella, yellow fever, bacterial antigens and any combination thereof.

In some embodiments, the biologically active macromolecule is an allergen selected from a group consisting of house dust mite, animal dander, molds, pollens, ragweed, latex, vespid venoms and insect-derived allergens, and any combination thereof.

The biologically active macromolecule may comprise a combination of macromolecules with similar biological functions, including, for example, a combination of a wild type molecule with its chemically or biologically modified analogs. Examples of such wild type macromolecules and analogs include, without limitation, glucagon-like peptide 1 (GLP-1) and its analogs, liraglutide, taspoglutide, albiglutide, lixisenatide and exenatide and its analogs, and any combination thereof.

In some embodiments, the therapeutic agent is selected from exenatide, a 39-amino acid peptide (marketed by Amylin and Eli Lilly as BYETTA® for the treatment of type 2 diabetes) and salts and functional derivatives such as pegylated exenatide, exenatide fusion proteins such as albumin, transferring, XTEN, Fc fusions, and fatty acid modified derivatives thereof.

### Permeation Enhancers

As noted above, a great number of permeation enhancers are known in the art. In some embodiments, the permeation enhancer is selected from the group consisting of a fatty acid, a medium chain glyceride, a surfactant, a steroidal detergent, an acyl carnitine, an alkanoyl choline, an N-acetylated amino acid, and esters, salts and derivatives thereof.

In some embodiments, the permeation enhancer comprises a fatty acid, including, without limitation, butyric, caproic, caprylic, pelargonic, capric, lauric, myristic, palmitic, stearic, arachidic, oleic, linoleic, linolinic acid, their salts, derivatives and any combination thereof. In some embodiments, the permeation enhancer comprises a glyceride of a fatty acid, including, without limitation, butyric, caproic, caprylic, pelargonic, capric, lauric, myristic, palmitic, stearic, arachidic, oleic, linoleic, linolinic acid, their salts, derivatives and any combination thereof. The glyceride may be a monoglyceride, a diglyceride, or triglyceride, and the fatty acid may comprise the same or different fatty acids. In some embodiments, the permeation enhancer comprises a fatty chain having 8 to 14 carbon atoms.

In some embodiments, the permeation enhancer comprises a bile acid or salt, including conjugated or un-conjugated bile acids, such as cholate, deoxycholate, taurocholate, glycocholate, taurodeoxycholate, ursodeoxycholate, tauroursodeoxycholate, chenodeoxycholate, their derivatives and combinations thereof.

In some embodiments, the permeation enhancer comprises a metal chelator, such as EDTA, or EGTA, a surfactant, such as sodium dodecyl sulfate, polyethylene ethers or esters, polyethylene glycol-12 lauryl ether, salicylate, polysorbate 80 (Tween 80®), nonylphenoxypolyoxyethylene, dioctyl sodium sulfosuccinate, saponin, palmitoyl carnitine, lauroyl 1-carnitine, dodecyl maltoside, acyl carnitines, alkanoyl choline, and any combination thereof. Other permeation enhancers include 3′-nitrobenzoate, zoonula occulden toxin, fatty acid ester of lactic acid salts, glycyrrhizic acid salt, hydroxyl beta-cyclodextrin, N-acetylated amino acids such as sodium N-[8-(2-hydroxybenzoyl)amino]caprylate, and chitosan, salts and derivatives of these compounds, and any combination thereof.

In some embodiments, the permeation enhancer comprises compounds that selectively target and open tight-junctions (e.g., chitosan and its derivatives). In some embodiments, the permeation enhancer is capric acid, or a salt or derivative thereof.

### Formulations and Excipients

As noted above, the present invention provides pharmaceutical compositions comprising, inter alia, a pharmaceutically acceptable excipient. The pharmaceutical composition may be in the form of capsules, tablets, pellets, patches, or discs. Suitable excipients and their formulations are known in the art and are described in REMINGTON: THE SCIENCE AND PRACTICE OF PHARMACY (21^{st} ed., Lippincott Williams & Wilkins, 2005), relevant sections of which are incorporated herein.

In some embodiments, the pharmaceutically acceptable excipients comprise the materials that assist the dispersion of carriers. In some embodiments, the pharmaceutical excipients comprise a disintegrant. In some embodiments, the pharmaceutical excipients comprise polyplasdone, croscarmellose, crospovidone, sodium starch glycolate, hydroxypropyl cellulose, and any combination thereof.

The pharmaceutical compositions may include other components, such as buffers, preservatives, nonionic surfactants, solubilizing agents, absorption enhancers, stabilizing agents, emollients, lubricants and tonicity agents. The composition may be formulated to achieve controlled release of the drugs.

The pharmaceutical compositions may be in a form suitable for oral use, for example, as tablets, troches, lozenges, or hard or soft capsules. Compositions intended for oral use may be prepared according to any method known in the art for the manufacture of pharmaceutical compositions and such compositions may contain one or more agents such as sweetening agents, flavoring agents, coloring agents and preserving agents, e.g. to provide pharmaceutically stable and palatable preparations.

The excipients used in a tablet may include, for example, inert diluents or fillers, such as calcium carbonate, sodium carbonate, lactose, calcium phosphate or sodium phosphate, microcrystalline cellulose, sucrose, mannitol, and sorbitol; granulating or binding agents such as polyvinyl pyrrolidone, polyethylene glycol, or hydroxypropyl methylcellulose, gelatin, starch, or a combination thereof; lubricating agents, for example, magnesium stearate, silica powder, stearic acid or talc. In the examplary formulations disclosed in this invention, the excipients comprising the core formulation include:

| Excipients | % Content | Function |
|---|---|---|
| Sodium Caprate | 40-80 | Permeation enhancer |
| Microcrystalline cellulose | 15-75 | Diluent |
| Hydroxypropyl methyl cellulose | 0-20% | Matrix |
| Mannitol | 0-30% | Diluent |
| Sodium carboxymethyl cellulose | 1-8% | Disintegrant |
| Polyvinyl pyrrolidone | 1-5% | Binder |
| Silica powder | 1-2% | Glidant |
| Magnesium stearate | 0.1-2% | Lubricant |
| Pharmaceutical ingredient | 0.1-20% | Therapeutic agent |

In some embodiments, the amount of permeation enhancer, particularly sodium caprate, in the formulation is about 400 mg, about 300 mg, about 200 mg, about 100 mg, about 50 mg, or about 25 mg, preferably about 200 mg, about 100 mg, or about 50 mg, and more preferably about 100 mg. In some embodiments, the content of sodium caprate ranges between about 25-300 mg, about 50-200 mg, or about 100-200 mg.

The presence of hydroxypropylmethyl cellulose (HPMC) in the core modulates the release profile of permeation enhancer and therapeutic agent adhesive properties of the core formulation. HPMC of various viscosity can be incorporated in proportion or in combination to allow modulate of adhesion of the core. The example >> illustrate

Macromolecular drugs incorporated in this invention are often highly potent, and the drug content in the core formulation is low, at about 1%, about 0.1% or about 0.01%. Low dose formulations such as these often face considerable challenges in quality control aspects, including stability, content uniformity, and analytical methods. It is often necessary to devise process to ensure stability of the therapeutic agent while maintain content uniformity.

High dose drug formulations tend to present different challenges compared with low dose formulations, such as difficulties in terms of compactability and flowability.

In some embodiments, the formulation may further comprise calcium phosphate nanoparticles as described in U.S. Patent Application Pub. No. 2005/0234114, U.S. Ser. No. 12/434,557, and PCT Pub. Nos. WO 2005/084637 and WO 2009/135190, all of which are incorporated herein by reference in their entireties.

The pharmaceutical compositions of the present invention may also be administered in the form of suppositories for rectal administration of the drug. These compositions can be prepared by mixing the pharmaceutical composition with a suitable non-irritating excipient which is solid at ordinary temperatures but liquid at the rectal temperature and will therefore melt in the rectum to release the drug. Such materials include, without limitation, cocoa butter and polyethylene glycols.

The pharmaceutical compositions of the present invention may also be administered through the vaginal route. Formulations suitable for vaginal administration include, without limitation, pessaries, tablets, or tampons.

The pharmaceutical compositions for the administration of the compounds of the invention may conveniently be presented in dosage unit form and may be prepared by any of the methods well known in the art of pharmacy.

### Bioadhesive Layers

As discussed above, the core pharmaceutical composition of the present invention is coated with a bioadhesive layer comprising a bioadhesive polymer. In some embodiments, the bioadhesive layer is coated on, and in direct contact with, the solid dosage form. In other embodiments, there may be one or more intermediate layers between the solid dosage form and the bioadhesive layer. In some embodiments, an impermeable or semi-permeable layer is positioned between the solid dosage form and the bioadhesive layer. Alternatively, the bioadhesive layer may be positioned between the solid dosage form and the impermeable or semi-permeable layer.

In some embodiments, the bioadhesive layer comprises a bioadhesive polymer that can adhere to the desired target site such as the gastrointestinal tract. The bioadhesive polymer may comprise, without limitation, carbomer, polycarbophil, chitosan, alginate, thiomer, gelatin, hydroxypropyl methylcellulose, carboxymethyl cellulose, hydroxyethyl cellulose, hydroxypropyl cellulose, polyvinyl alcohol, polyethylene glycol, polyvinyl pyrrolindone, fumaric anhydride oligomer, polyesters, polyacrylates, polysaccharides, modified dextrans, sodium hyaluronate, pectin, xanthan gum, as well as their salts, derivatives and mixtures.

In preferred embodiments, the bioadhesive layer comprises hydroxypropyl methylcellulose (HPMC) or polycarbophil AA1.

In some embodiments, the bioadhesive layer comprises a composite of materials, such as a bioadhesive polymer, a plasticizer, or other materials to module the release rate. It is desirable to have high content of bioadhesive materials. In some embodiments, the content of a bioadhesive polymer comprises above about 50%, above about 65%, above about 75%, above about 80%, or above about 90%.

In some embodiments, the bioadhesive layer may further comprise an enteric material, such as cellulose acetate phthalate (EUDRAGIT® S or L), hydroxypropyl methylcellulose phthalate, hydroxypropyl methylcellulose acetate succinate, cellulose acetate trimellitate, polyvinyl acetate phthalate, methacrylic acid copolymers, shellac, their salts and derivatives, and any combination thereof.

The presence of enteric material in the bioadhesive layer may preserve integrity of the dosage in the stomach and allow release and absorption in the intestines where pH is raised. It often requires high proportion of enteric material such as about 50% or more in order to maintain acid stability. It is therefore quite surprising to observe the acid stability of the dosage even with enteric material as low as about 10-20%.

In some embodiments, the content of enteric material contained in the bioadhesive layer ranges between about 5-50%, preferably about 10-35%, and more preferably about 15-25%.

In some embodiments, the bioadhesive layer may further comprise a plasticizer, such as glycerol, triacetin, polyvinyl alcohol, polyethylene glycol, their derivatives, and any combination thereof. In some embodiment, the plasticizer in the bioadhesive layer ranges between about 1-35%, preferably about 5-25%, and more preferably about 10-15%.

In some embodiments, the bioadhesive layer can be present in a content ranging from about 0.5% to about 10%, preferably about 1-5%, more preferably about 2-3% by weight of the pharmaceutical composition.

In some embodiments, the thickness of the bioadhesive layer can be modulated to achieve desirable release kinetics and absorption.

The enteric material dissolves quickly once the desired pH is reached. Since the amounts of enteric material presented in the bioadhesive and enteric layers in the composition do not differ significantly, the dissolution rates between a bioadhesive layer containing enteric material and an enteric layer would be expected to be similar.

Therefore, it is quite unexpected to observe a faster absorption and pharmacological effect from a formulation with a bioadhesive layer containing enteric material compared with a formulation containing an enteric layer containing the same enteric material.

This surprising finding suggests that formulations with enteric material incorporated into the bioadhesive layer may allow an increased absorption window in the intestines and can achieve earlier clinical responses, which is generally more desirable.

The formulations with enteric material incorporated into the bioadhesive layer reduce the coating requirements and simplify the manufacturing process, thereby reducing the production time and subsequently the cost to produce the desired formulations.

### Impermeable or Semi-Permeable Layers

As discussed above, the impermeable or semi-permeable layer is used to modulate the release kinetics and to further reduce the amount of permeation enhancers required to improve the absorption and bioavailability of poorly absorbed therapeutic agents. In some embodiments, the impermeable or semi-permeable layer comprises an opening that is capable of directing a substantially unidirectional release of the therapeutic agent and the permeation enhancer from the solid dosage form. The size and shape of the opening will necessarily depend on the nature of the solid dosage form and the desired release kinetics. Determining the size of the opening is well within the skill of a person skilled in the art. In some embodiments, the opening may fully cover a single side of a tablet or caplet.

In some embodiments, the opening of the unidirectional release tablet covers about 20-90%, preferably about 40-80%, and more preferably about 50-70% of the area on one side of the tablet.

The of the unidirectional release tablet opening of the unidirectional release tablet can have any shape, for example, a circle, a triangle, a square, a rectangle, a rhombus, a parallelogram, a trapezium, or any other shape.

In some embodiments, the impermeable or semi-permeable layer comprises one or more hydrophilic polymers and/or one or more hydrophobic polymers. Examples of hydrophilic polymers include, without limitation, protein-based polymers (for example, gelatin or casein), pectin, agarose (agar), chitosan, carrageenan, starch, dextran, methylcellulose, calcium carboxymethyl cellulose, sodium carboxymethyl cellulose, cross-linked polymers of sodium carboxymethyl cellulose (for example, croscarmellose sodium), microcrystalline cellulose, ethylcellulose, hydroxyethyl cellulose, hydroxypropyl cellulose, hydroxypropyl methylcellulose, hydroxypropyl methylcellulose phthalate, cellulose ethers, cellulose acetate, cellulose acetate phthalate, other cellulose derivatives, polyvinyl alcohol, polyvinylpyrrolidone (PVP), cross-linked povidone, other vinyl polymers and copolymers, guar gum, poloxamer, polyethylene glycol, polyethylene oxide, polyacrylic acid, polyethers, alkoxy polymers, sodium alginate, xanthan gum, other natural hydrogels or hydrogels derived from natural products, or a combination thereof. Examples of hydrophobic polymers include, without limitation, ethyl cellulose, polyester (such as polycaprolactone (PCL), polyesteramide (PEA), polyhydroxyalkanoate (PHA), polylactic acid (PLA), polylatic glycolic acid (PLGA), polyhydroxybutyrate-co-hydroxyvalerate (PHBV) and polybutylene succinate adipate (PBSA)), wax and low melting wax, polyethylene and ethylene copolymers, ethylene/vinyl acetate, polypropylene, polyurethane, ethylene/vinyl alcohol, polyvinyl alcohol, polyvinyllidene, polyolefin, or any combination thereof.

In some embodiments, the impermeable or semi-permeable layer further contains a solvent or plasticizer which makes the layer more flexible. The solvent may be any solvent that is compatible with the impermeable or semi-permeable material, including, for example, water and ethanol. Examples of plasticizers include, without limitation, citrate esters (e.g., triethylcitrate, triacetin), low molecular weight polyalkylene oxides (e.g., polyethylene glycols, polypropylene glycols, polyethylene/propylene glycols), glycerol, pentaerythritol, glycerol monoacetate, diacetate or triacetate, propylene glycol, sodium diethyl sulfosuccinate, sugar alcohols, corn syrup, and any combination thereof.

In some embodiments, the impermeable layer comprises an ethyl cellulose polymer in conjunction with a glycerol, polyethylene glycol, or triacetin as plasticizer. In some embodiments, the semi-permeable layer comprises a cellulose acetate in conjunction with polyethylene glycol, triacetin or glycerol as plasticizer.

In some embodiments, the content of plasticizer in a impermeable or semi-permeable layer ranges from 5-40%, preferably 10-30%, or more preferably 15-25%.

In some embodiments, the impermeable or semi-permeable layer can be present in a content ranging from about 0.5% to about 10%, preferably about 1-5%, more preferably about 2-4% by weight of the pharmaceutical composition.

The impermeable layer may further include other components, such as antiseptic agents, preservatives, and other ingredients to improve the stability of the pharmaceutical composition. Examples of additional plasticizers and components may be found in M. & I. Ash, THE HANDBOOK OF PHARMACEUTICAL ADDITIVES (3^{rd} ed., Synapse Information Resources, Inc., 2007), relevant sections of which are incorporated herein.

In some embodiments, the formation of a unidirectional opening on a formulation is achieved by a laser ablation process commonly used in the production of osmotic pumps. Unlike osmotic pump tablets, the unidirectional aperture is considerably larger (3-9 mm diameter compared to less than 0.5 mm diameter for a typical 10 mm diameter tablet), and the coating is much thinner. Therefore, different laser source and equipment configurations have to be adapted for the present invention.

### Enteric Layers

In some embodiments, the pharmaceutical composition in this invention comprises a core formulation coated with a bioadhesive layer, and a unidirectional layer may be further coated with a site-selective agent to allow release of the drugs at selected sites in the gastrointestinal tract. In some embodiments, the site-selective agent comprises a pH sensitive polymer that can dissolve in an environment with certain pH values. Coating with the site-selective agent allows the carrier to selectively expose the adhesive layer to certain regions of the gastrointestinal tract.

The enteric coating polymer may be selected, without limitation, from cellulose acetate phthalate, EUDRAGIT® S or L, hydroxypropyl methylcellulose phthalate, hydroxypropyl methylcellulose acetate succinate, cellulose acetate trimellitate, polyvinyl acetate phthalate, methacrylic acid copolymers, shellac, their salts and derivatives, and any combination thereof. In one particularly preferred embodiment, the enteric coating polymer is EUDRAGIT® L30D-55.

In some embodiments, the site-selective agent comprises polymers that can selectively attach to or release in the colon. Such colon selective agents include, but are not limited to, azo polymers and colon degradable polysaccharides such as pectin, amylose, guar gum, xylan, cyclodextrin, dextran, their salts and derivatives, and any combination thereof.

The thickness of the coating is selected to provide the desired release rate, which is dependent on both the nature and thickness of the coating. In some embodiments, the enteric layer comprises about 1 to 15%, more preferably about 3 to 12%, and most preferably about 6 to 10% by weight based on the combined weight of the solid dosage form and the coating.

### Exemplary Formulations

In view of the foregoing, it is understood that the present invention contemplates a variety of solid dosage forms including many different combinations of therapeutic agents, permeation enhancers, bioadhesive layers, semi-permeable or impermeable layers, and/or enteric layers.

In some embodiments, the solid dosage form includes an exendin or an exendin peptide analog as the therapeutic agent and sodium caprate as the permeation enhancer; the bioadhesive layer includes HPMC or AA1 as the bioadhesive polymer and EUDRAGIT® L30D-55 as the enteric polymer; and the semi-permeable layer includes cellulose acetate or ethyl cellulose and an opening allowing unidirectional release. In some embodiments, the bioadhesive layer comprises at least about 70% bioadhesive polymer by weight of the layer and at least about 5% enteric polymer by weight of the layer. In some embodiments, the amount of sodium caprate ranges between about 100 and 150 mg. In some embodiments, the exendin is exendin-4, and the exendin peptide analog is exenatide or one of its salts or functional derivatives. In some embodiments, the bioadhesive layer includes HPMC as the bioadhesive polymer; the semi-permeable layer includes cellulose acetate and an opening allowing unidirectional release; and the solid dosage is in the form of a tablet. In some embodiments, the bioadhesive layer includes HPMC as the bioadhesive polymer; the semi-permeable layer includes ethyl cellulose and an opening allowing unidirectional release; and the solid dosage is in the form of a tablet.

In some embodiments, the solid dosage form includes an exendin or an exendin peptide analog as the therapeutic agent and sodium caprate as the permeation enhancer; the bioadhesive layer includes HPMC or AA1 as the bioadhesive polymer; the semi-permeable layer includes cellulose acetate or ethyl cellulose and an opening allowing unidirectional release; and the enteric layer includes EUDRAGIT® L30D-55. In some embodiments, the bioadhesive layer comprises at least about 70% bioadhesive polymer by weight of the layer. In some embodiments, the amount of sodium caprate ranges between about 100 and 150 mg. In some embodiments, the exendin is exendin-4, and the exendin peptide analog is exenatide or one of its salts or functional derivatives. In some embodiments, the bioadhesive layer includes HPMC as the bioadhesive polymer; the semi-permeable layer includes cellulose acetate and an opening allowing unidirectional release; and the solid dosage is in the form of a tablet. In some embodiments, the bioadhesive layer includes HPMC as the bioadhesive polymer; the semi-permeable layer includes ethyl cellulose and an opening allowing unidirectional release; and the solid dosage is in the form of a tablet.

### Production Methods

In one aspect, the present invention provides a method for making the present pharmaceutical composition comprising of the following steps: fabricating a solid dosage form comprising an effective amount of a therapeutic agent, a permeation enhancer and a pharmaceutically acceptable excipient; coating the solid dosage form with a bioadhesive layer comprising a bioadhesive polymer; and optionally coating the solid dosage form with an impermeable or semi-permeable layer comprising an opening capable of directing a substantially unidirectional release of the therapeutic agent and the permeation enhancer from the solid dosage form. In some embodiments, the order of applying the bioadhesive polymer layer and the impermeable or semi-permeable layer is reversed. In some embodiments, the method further comprises coating the composition with an enteric layer.

In some embodiments, an aqueous suspension of bioadhesive and impermeable or semi-permeable materials such as ethyl cellulose or EUDRAGIT® L30-D55 may be used. In some embodiments, the materials may be dispensed directly without solvent. In some embodiments, polycaprolactone (PCL) or wax may be directly dispensed when heated.

In a preferred embodiment, the pharmaceutical composition of present invention is produced in the form of tablets or caplets. The core tablets are formed by compression, commonly achieved with a rotary press. The tablet fabrication processes, including wet granulation and direct compression, have been amply described and are well known art. (DEVELOPING SOLID ORAL DOSAGE FORMS: PHARMACEUTICAL THEORY AND PRACTICE, Ed. by Qiu et al., Academic Press 2009).

However, the development of tablet form for macromolecular drugs remains challenging. Most macromolecular drugs are peptides and proteins, which have delicate structure and are highly unstable. These macromolecular drugs often are highly potent and thus require low dose formulations, which require special processes such as wet granulation to ensure content of uniformity. (FORMULATION AND ANALYTICAL DEVELOPMENT FOR LOW DOSE ORAL DRUG PRODUCTS, Ed. by Zheng, Wiley 2009).

The difficulty of process development is illustrated in an example with exenatide and insulin as drug. When wet granulation process is used to produce the tablets, it is found that the content of uniformity is indeed within specifications yet exenatide is unstable, while exenatide powder and direct blend with exenatide and excipients in the formulations maintain stability under the same conditions.

Therefore, direct compression process is an appropriate choice for drugs of acceptable dose such as about 0.5-2 mg. The direct compression process in common low dose formulation often is straightforward since the content of the drug is so low that the properties of the tablet such as compactability, flowability and hardness are not affected by the presence of the drug as long as uniformity of content can be assured. For example, insulin powder, which exhibits small crystal particles and is less hydroscopic, can be incorporated into a direct compression process to manufacture tablets when the particle size of the drug powder is properly controlled.

The formulation in the present invention presents yet another challenge due to the presence of large proportion of permeation enhancer such as sodium caprate, which exhibits poor compactability and may also cause particle segregation. In some embodiments, the direct compression formulation in the present invention is illustrated in the table below.

| Excipients | % Content | Function |
|---|---|---|
| Sodium Caprate | 40-80 | Permeation enhancer |
| Microcrystalline cellulose | 15-75 | Diluent |
| Hydryoxypropyl methyl cellulose | 0-10% | Matrix |
| Soldium carboxymethyl cellulose | 1-8% | Disintegrant |
| Silica powder | 1-2% | Glidant |
| Magnesium stearate | 0.1-2% | Lubricant |
| Pharmaceutical ingredient | 0.1-20% | Therapeutic agent |

For drugs like exenatide, the unit dose is expected to be even lower and/or when drug powder exhibits hydroscopic property, direct compression process is often not recommended due to difficulties in achieving content of uniformity in manufacturing process (Zheng 2009, ibid.)

In the present invention, the balance between stability and content uniformity can be maintained by a non-solvent granulation process. In some embodiments, the drug powder is suspended in a fluid medium in which drug is insoluble and the drug particle size can be controlled and monitored by sonication or homogenization. Binder may be added in the suspension to promote homogeneity. The suspension is added to the excipient mixture as in the commonly practiced granulation process. The non-solvent granulation process can help to maintain stability while ensuring content uniformity, as illustrated in Examples 12 and 13.

In some embodiments, the commonly used non-solvents that can be used in the present invention include ethanol, isopropanol, acetone, and ethyl acetate.

### Methods of Use

The pharmaceutical composition of the present invention is useful for delivering a drug to a desired mucosal surface. The composition may selectively attach to a mucosal surface, and the therapeutic agent and permeation enhancer contained in the solid dosage form will flow from the carrier to the mucosal surface unidirectionally to generate high local concentrations of both. Thus, the present pharmaceutical composition can enhance the absorption of drug with a low amount of permeation enhancer that is otherwise ineffective.

Accordingly, in one aspect, the present invention provides a method of delivering a therapeutic using the pharmaceutical composition. In some embodiments, the present invention provides a method of treating a subject in need of a therapeutic treatment, comprising administering to the subject the pharmaceutical composition disclosed herein, preferably to a mucosal surface. The pharmaceutical compositions may be administered to the subject by any means known in the art, including, without limitation, oral, buccal, sublingual, vaginal, and rectal routes. Administration may be systemic or localized.

### EXAMPLES

### Example 1

### Fabrication of Tablets

1.1 Core Tablet Fabrication

The core tablets were fabricated according to the formula listed in Table 1 by compressing the materials with a single tablet press. All the components except exenatide and magnesium stearate were first weighed and mixed thoroughly. Granules were then formed with 15% polyvinylpyrrolidone (PVP) in 25% ethanol as adhesive material and dried at 60° C. for 2 hrs. The granules were sieved through a 22-mesh screen and weighed based on the single tablet along with exenatide and magnesium stearate. The composition was mixed and pressed into tablet. All tablets were weighed individually, and those tablets with more than 5% of the average weight were excluded.

TABLE 1
Core tablet formulations (amounts shown in milligrams).

| # | Exenatide | SCA | MCC | Mannitol | HPMC | PVP | MST | Silica | Total |
|---|---|---|---|---|---|---|---|---|---|
| 1 | 3 | 50 | 70 | 156 | 15 | 1.5 | 1.5 | 3 | 300 |
| 2 | 3 | 100 | 86 | 90 | 15 | 1.5 | 1.5 | 3 | 300 |
| 3 | 3 | 200 | 31 | 45 | 15 | 1.5 | 1.5 | 3 | 300 |
| 4 | 3 | 400 | 65 | 90 | 30 | 3 | 3 | 6 | 600 |
| 5 | 5 | 500 | 130 | | | 7.5 | | 7.5 | 650 |

SCA—sodium caprate; MCC—microcellulose crystalline (Avicel PH-101); PVP—polyvinyl pyrrolidone (PVP K30); HPMC—hydroxypropyl methylcellulose; MST—magnesium stearate; silica—silica powder.

1.2 Bioadhesive Layer

The core tablets were further coated with bioadhesive polymers, hydroxypropyl methylcellulose (HPMC) or polycarbophil AA1. For HPMC coating, the tablets were coated with a 2% HPMC aqueous solution in a small scale tablet coating machine (BY300A, Yellow Sea Machinery). The weight gain due to the coating was 2% of the tablet weight Separately, the tablets were coated with 4% polycarbophil AA1 in ethanol with a small tablet coating machine. The weight gain was 3%. Coated tablets were dried at 30° C. for 14 hrs.

1.3 Unidirectional Release Layer

The core tablets were subsequently coated with a layer comprising either impermeable or semi-permeable materials. The tablets were first covered on one side with adhesive paper to create unidirectional release openings. Next, the tablets were coated with either 4% ethyl cellulose in ethanol containing 20% triacetin as plasticizer or 3% cellulose acetate in a mixture of acetone and formic acid (9:1 v/v) containing 20% polyethylene glycol, MW 2000 (PEG 2000) as plasticizer. After coating, the tablets were dried at 30° C. for 30 min. The weight gain due to the coating was adjusted between 2-5%. Finally, the adhesive paper was peeled off to expose the unidirectional release openings.

1.4 Enteric Layer

The tablets were coated with enteric polymer EUDRAGIT® L30D-55. The coating mixture contained 200 g L30D-55, 12 g ultrafine talc powder and 6 g polyethylene glycol, MW 6000 (PEG 6000), homogenized in water to a final volume of 400 ml. The coating was performed in a small scale tablet coating machine. The weight gain due to the enteric coating was 10% of the tablet weight. The tablets were dried at 30° C. for 14 hrs.

### Example 2

### Synchronous Release of Exenatide and Sodium Caprate

Kinetic release profiles of exenatide and sodium caprate in different formulations were evaluated in several in vitro tests.

First, acid susceptibility of the formulations was tested by placing enteric coated tablets in 100 ml 0.1N HCl at 37° C. for 2 hrs in a drug dissolution apparatus. Samples were taken at various time points, and the concentrations of exenatide and sodium caprate were determined using an HPLC system with a C18 column (Waters). Tablets were found intact in acid media and no exenatide or sodium caprate was detected.

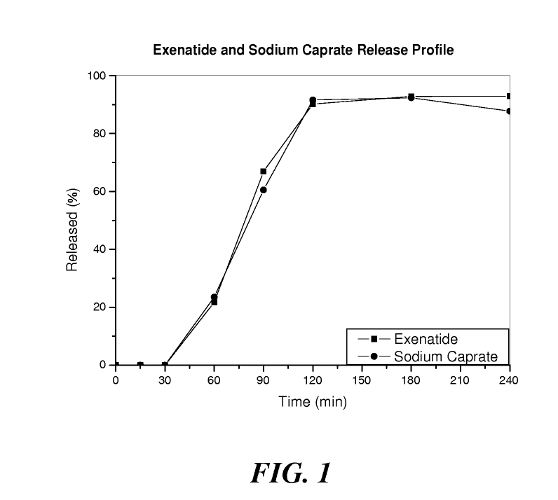

Kinetic release profiles were further studied by removing the acid media and replacing it with 100 ml simulated intestinal fluid, pH 6.8. The release was monitored at 37° C., and samples were taken at various time points to determine the concentration of exenatide or sodium caprate. Table 2 shows the fractions of exenatide or sodium caprate released from the enteric and HPMC coated tablets containing 50 mg sodium caprate. The results are also summarized in FIG. 1. As one can easily see, the release of exenatide and sodium caprate at pH 6.8 is substantially synchronous.

TABLE 2
Release of exenatide and sodium caprate
from HPMC and enteric coated tablets.

| Time (min) | Exenatide Release (%) | Sodium Caprate Release (%) |
|---|---|---|
| 0 | 0 | 0 |
| 15 | 0 | 0 |
| 30 | 0 | 0 |
| 60 | 21.73 | 23.54 |
| 90 | 66.91 | 60.48 |
| 120 | 90.15 | 91.55 |
| 180 | 92.82 | 92.36 |
| 240 | 92.85 | 87.72 |

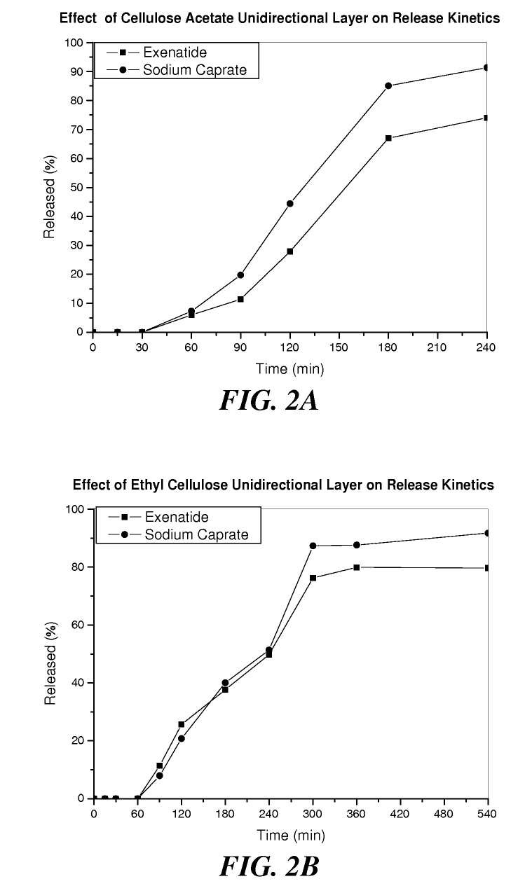

Similarly, Table 3 shows the fractions of exenatide or sodium caprate released from the tablets coated with enteric polymer, cellulose acetate, and HPMC containing 50 mg sodium caprate. The results are also summarized in FIG. 2A. Once again, the release of exenatide and sodium caprate is substantially synchronous, and yet significantly extended compared to the tablets not containing a cellulose acetate layer (maximum release reached at 3 hours vs. 2 hrs).

TABLE 3
Release of exenatide and sodium caprate from HPMC,
enteric and cellulose acetate (CA) coated tablets.

| Time (min) | Exenatide Release (%) | Sodium Caprate Release (%) |
|---|---|---|
| 0 | 0 | 0 |
| 15 | 0 | 0 |
| 30 | 0 | 0 |
| 60 | 6.05 | 7.30 |
| 90 | 11.41 | 19.75 |
| 120 | 27.91 | 44.40 |
| 180 | 66.99 | 85.07 |
| 240 | 74.01 | 91.34 |

Table 4 shows the fractions of exenatide or sodium caprate released from the tablets coated with enteric polymer, ethyl cellulose, and HPMC containing 50 mg sodium caprate. The results are also summarized in FIG. 2B. As in the previous experiment, the release of exenatide and sodium caprate is substantially synchronous. Notably, release from the ethyl cellulose coated tablets is further extended compared to the tablets containing a cellulose acetate layer (maximum release reached at 5 hrs vs. 3 hrs).

TABLE 4
Release of exenatide and sodium caprate from HPMC,
enteric and ethyl cellulose (EC) coated tablets.

| Time (min) | Exenatide Release (%) | Sodium Caprate Release (%) |
|---|---|---|
| 0 | 0 | 0 |
| 15 | 0 | 0 |
| 30 | 0 | 0 |
| 60 | 0 | 0 |
| 90 | 11.43 | 7.96 |
| 120 | 25.65 | 20.78 |
| 180 | 37.63 | 40.05 |
| 240 | 49.75 | 51.29 |
| 300 | 76.22 | 87.28 |
| 360 | 79.85 | 87.58 |
| 540 | 79.64 | 91.70 |

### Example 3

### Effect of Bioadhesive Layer on Exenatide Absorption in Dogs with 100 mg Sodium Caprate

Absorption of exenatide in different formulations containing 100 mg sodium caprate as permeation enhancer was evaluated in healthy beagles.

Twelve beagle dogs with body weights between 8-12 kg were housed in an animal facility (Shanghai TCM University Animal Center). Water was supplied ad libitum. The dogs were randomly divided into six groups, and repeated treatments were performed with 1 week resting period. The dogs were fasted overnight, and the tablets were fed directly with 10 ml water. Food was restricted until 6 hrs after dosing. The treatment groups evaluated in this experiment are summarized in Table 5.

TABLE 5
Treatment groups for evaluating the effect of a bioadhesive
layer on the bioavailability of exenatide.

| Group | Group | |
|---|---|---|
| # | ID | Treatment |
| 1 | Blank | Placebo tablet |
| 2 | SC | Subcutaneous injection of exenatide, 60 μg/dog |
| | | in 10 mM sodium acetate, pH 4.0 |
| 3 | No coating | 3 mg exenatide and 100 mg sodium caprate in |
| | | enteric coated tablet |
| 4 | HPMC layer | 3 mg exenatide and 100 mg sodium caprate in |
| | | HPMC and enteric coated tablet |
| 5 | AA1 layer | 3 mg exenatide and 100 mg sodium caprate in |
| | | AA1 and enteric coated tablet |
| 6 | Chitosan layer | 3 mg exenatide and 100 mg sodium caprate in |
| | | chitosan and enteric coated tablet |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

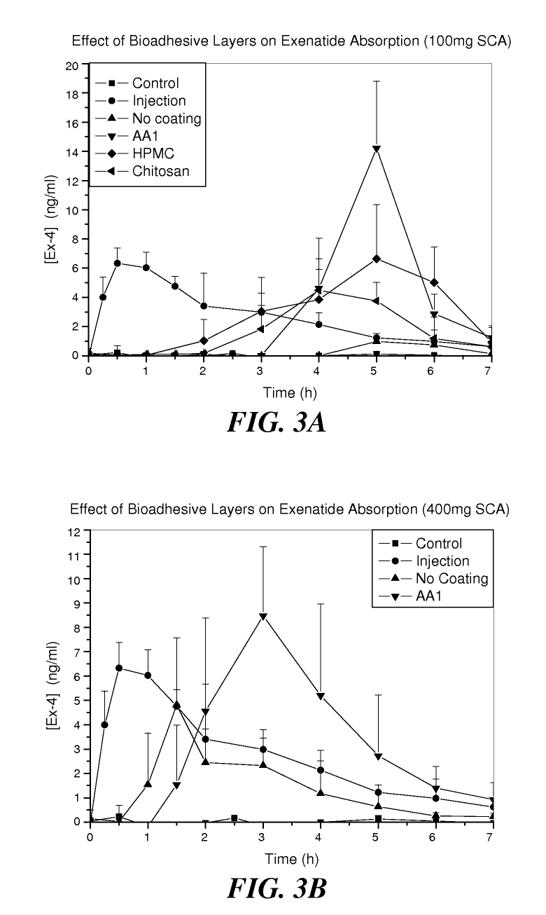

All the samples including standards were adjusted to contain 20% serum. The serum concentrations of exenatide were calculated based on the standard curve and area under curve was estimated and compared. The relative bioavailabilities and kinetics of exenatide absorption for each group are summarized in Table 6 and FIG. 3A.

TABLE 6
Effect of a bioadhesive layer on the bioavailability of
exenatide in the presence of 100 mg sodium caprate.

| Group ID | N | Bioavailability (%) |
|---|---|---|
| Blank | 3 | 0 |
| SC | 3 | 100 |
| No coating | 3 | 0.19 |
| HPMC layer | 3 | 2.12 |
| AA1 layer | 3 | 2.37 |
| Chitosan layer | 3 | 1.25 |

The results indicate that absorption of exenatide is significantly affected by the bioadhesive layer. Without a bioadhesive layer, exenatide absorption is minimal in the presence of 100 mg sodium caprate as permeation enhancer. The coating with AA1, HPMC, or chitosan significantly improves exenatide absorption, although the effect of chitosan is significantly less pronounced.

### Example 4

### Effect of Bioadhesive Layer on Exenatide Absorption in Dogs with 400 mg Sodium Caprate

The absorption of exenatide in different formulations containing 400 mg sodium caprate was evaluated in healthy beagles.

Twelve beagle dogs with body weights between 8-12 kg were housed in an animal facility (Shanghai TCM University Animal Center). Water was supplied ad libitum. The dogs were randomly divided into four groups, and repeated treatments were performed with 1 week resting period. The dogs were fasted overnight, and the tablets were fed directly with 10 ml water. Food was restricted until 6 hrs after dosing. The treatment groups evaluated in this experiment are summarized in Table 7.

TABLE 7
Treatment groups for evaluating the effect of a bioadhesive
layer on the bioavailability of exenatide.

| Group | Group | |
|---|---|---|
| # | ID | Treatment |
| 1 | Blank | Placebo tablet |
| 2 | SC | Subcutaneous injection of exenatide, 60 μg/dog |
| | | in 10 mM sodium acetate, pH 4.0 |
| 3 | No coating | 5 mg exenatide and 500 mg sodium caprate in |
| | | enteric coated tablet |
| 4 | AA1 layer | 3 mg exenatide and 400 mg sodium caprate in |
| | | AA1 and enteric coated tablet |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

All the samples including standards were adjusted to 20% serum. The serum concentrations of exenatide were calculated based on a standard curve, and areas under the curve were estimated and compared. The relative bioavailabilities and kinetics of exenatide absorption for each group are summarized in Table 8 and FIG. 3B.

TABLE 8
Effect of a bioadhesive layer on the bioavailability of
exenatide in the presence of 400 mg sodium caprate.

| Group ID | N | Bioavailability (%) |
|---|---|---|
| Blank | 3 | 0 |
| SC | 3 | 100 |
| No coating | 3 | 0.61 |
| AA1 layer | 3 | 2.34 |

As in Example 3, the results indicate that absorption of exenatide is significantly affected by the bioadhesive layer. Without a bioadhesive layer, exenatide absorption is moderate in the presence of 500 mg sodium caprate as permeation enhancer. The coating with AA1 significantly improves exenatide absorption.

### Example 5

### Effect of Amount of Sodium Caprate on Exenatide Absorption in Dogs

The absorption of exenatide in formulations containing different amount of sodium caprate was evaluated in healthy beagles.

Twelve beagle dogs with body weights between 8-12 kg were housed in an animal facility (Shanghai TCM University Animal Center). Water was supplied ad libitum. The dogs were randomly divided into six groups, and repeated treatments were performed with 1 week resting period. The dogs were fasted overnight, and the tablets were fed directly with 10 ml water. Food was restricted until 6 hrs after dosing. The treatment groups evaluated in this experiment are summarized in Table 9.

TABLE 9
Treatment groups for evaluating the effect of sodium
caprate amount on the bioavailability of exenatide.

| Group | Group | |
|---|---|---|
| # | ID | Treatment |
| 1 | Blank | Placebo tablet |
| 2 | SC | Subcutaneous injection of exenatide, 60 μg/dog |
| | | in 10 mM sodium acetate, pH 4.0 |
| 3 | 50 mg | 3 mg exenatide and 50 mg sodium caprate in |
| | | AA1 and enteric coated tablet |
| 4 | 100 mg | 3 mg exenatide and 100 mg sodium caprate in |
| | | AA1 and enteric coated tablet |
| 5 | 200 mg | 3 mg exenatide and 200 mg sodium caprate in |
| | | AA1 and enteric coated tablet |
| 6 | 400 mg | 3 mg exenatide and 400 mg sodium caprate in |
| | | AA1 and enteric coated tablet |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

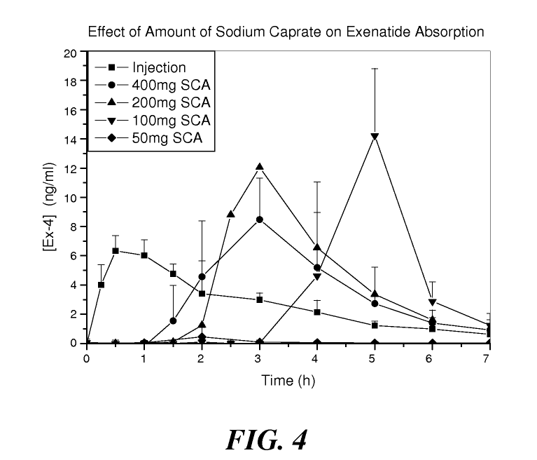

All the samples including standards were adjusted to 20% serum. The serum concentrations of exenatide were calculated based on a standard curve, and areas under the curve were estimated and compared. The relative bioavailabilities and kinetics of exenatide absorption for each group are summarized in Table 10 and FIG. 4.

TABLE 10
Effect of sodium caprate amount on the bioavailability
of exenatide in the presence of AA1 and enteric layer.

| Group ID | N | Bioavailability (%) |
|---|---|---|
| Blank | 3 | 0 |
| SC | 3 | 100 |
| 50 mg | 3 | 0.08 |
| 100 mg | 3 | 2.37 |
| 200 mg | 3 | 2.62 |
| 400 mg | 3 | 2.34 |

The results indicate that absorption of exenatide is dependent on the amount of sodium caprate. Exenatide absorption is minimal in the presence of 50 mg sodium caprate as permeation enhancer, whereas significant exenatide absorption is observed for sodium caprate above 100 mg. No further improvement was seen above 100 mg sodium caprate.

### Example 6

### Effect of Unidirectional Release Dosage on Exenatide Absorption in Dogs

The absorption of exenatide in formulations containing 50 mg sodium caprate was evaluated in healthy beagles.

Twelve beagle dogs with body weights between 8-12 kg were housed in an animal facility (Shanghai TCM University Animal Center). Water was supplied ad libitum. The dogs were randomly divided into five groups, and repeated treatments were performed with 1 week resting period. The dogs were fasted overnight, and the tablets were fed directly with 10 ml water. Food was restricted until 6 hrs after dosing. The treatment groups evaluated in this experiment are summarized in Table 11.

TABLE 11
Treatment groups for evaluating the effect of a unidirectional
release layer on the bioavailability of exenatide.

| Group | Group | |
|---|---|---|
| # | ID | Treatment |
| 1 | Blank | Placebo tablet |
| 2 | SC | Subcutaneous injection of exenatide, 60 μg/dog in |
| | | 10 mM sodium acetate, pH 4.0 |
| 3 | No | 3 mg exenatide and 50 mg sodium caprate in HPMC |
| | coating | and enteric coated tablet |
| 4 | CA | 3 mg exenatide and 50 mg sodium caprate in HPMC |
| | layer | and enteric coated tablet further coated with |
| | | cellulose acetate |
| 5 | EC | 3 mg exenatide and 50 mg sodium caprate in HPMC |
| | layer | and enteric coated tablet further coated with |
| | | ethyl cellulose |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

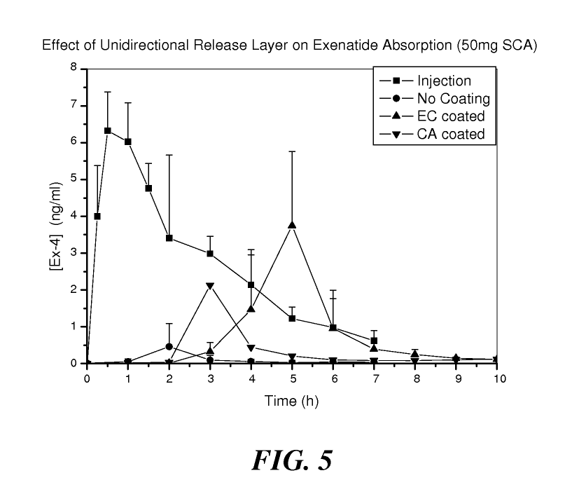

All the samples including standards were adjusted to 20% serum. The serum concentrations of exenatide were calculated based on the standard curve and area under curve was estimated and compared. The relative bioavailabilities and kinetics of exenatide absorption for each group are summarized in Table 12 and FIG. 5.

TABLE 12
Effect of a unidirectional release layer on the bioavailability
of exenatide in the presence of 50 mg sodium caprate and HPMC.

| Group ID | N | Bioavailability (%) |
|---|---|---|
| Blank | 3 | 0 |
| SC | 3 | 100 |
| No coating | 3 | 0.08 |
| CA layer | 3 | 0.34 |
| EC layer | 3 | 0.78 |

The results indicate that absorption of exenatide can be further enhanced by using a unidirectional release layer in conjunction with a bioadhesive layer. Without a unidirectional release layer, exenatide absorption and bioavailability in the presence of 50 mg sodium caprate and HPMC are minimal. Coating the tablet with a cellulose acetate semi-permeable membrane or an ethyl cellulose impermeable membrane containing an opening on one face of the tablet significantly improves exenatide absorption using the same amount of sodium caprate and HPMC.

### Example 7

### Bioavailability of Oral Exenatide in Dogs

The absorption of exenatide in formulations containing 100 mg sodium caprate was evaluated in healthy beagles.

Twelve beagle dogs with body weights between 8-12 kg were housed in an animal facility (Shanghai TCM University Animal Center). Water was supplied ad libitum. The dogs were randomly divided into three groups, and repeated treatments were performed with 1 week resting period. The dogs were fasted overnight, and the tablets were fed directly with 10 ml water. Food was restricted until 6 hrs after dosing. The treatment groups evaluated in this experiment are summarized in Table 13.

TABLE 13
Treatment groups for evaluating the bioavailability
of oral exenatide in dogs.

| Group | Group | |
|---|---|---|
| # | ID | Treatment |
| 1 | Blank | Placebo tablet |
| 2 | SC | Subcutaneous injection of exenatide, 60 μg/dog in |
| | | 10 mM sodium acetate, pH 4.0 |
| 3 | EC | 1 mg exenatide and 100 mg sodium caprate in HPMC |
| | layer | and enteric coated tablet further coated with |
| | | ethyl cellulose |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

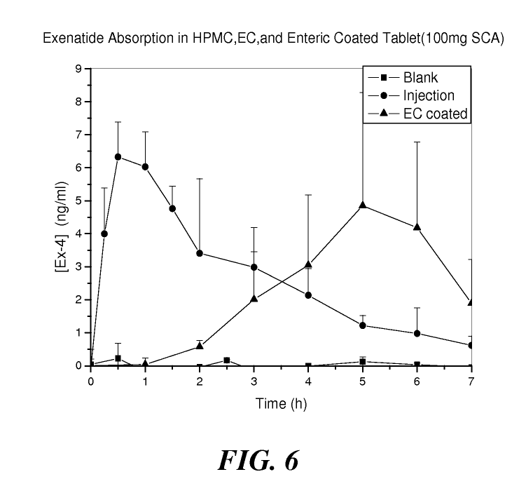

All the samples including standards were adjusted to 20% serum. The serum concentrations of exenatide were calculated based on the standard curve and area under curve was estimated and compared. The relative bioavailabilities and kinetics of exenatide absorption for each group are summarized in Table 14 and FIG. 6.

TABLE 14
Bioavailability of oral exenatide in the presence of 100 mg
sodium caprate, HPMC, ethyl cellulose and enteric coating.

| Group ID | N | Bioavailability (%) |
|---|---|---|
| Blank | 3 | 0 |
| SC | 3 | 100 |
| EC layer | 3 | 4.98 |

The results indicate that relative bioavailability of oral exenatide in the form of HPMC, ethyl cellulose and enteric coated tablet in the presence of 100 mg sodium caprate is 4.98%, compared with subcutaneous injection. Coating the tablet with an ethyl cellulose impermeable membrane containing an opening on one face of the tablet significantly improves exenatide absorption and relative bioavailability using the same amount of sodium caprate and HPMC (2.37%; see Example 5, Group 4).

### Example 8

### Effect of Enteric Layer on Exenatide Absorption in Dogs

8.1 Core Tablet Fabrication

The core tablets were fabricated according to the formula listed in Table 15 by compression using a single tablet press. All the components except exenatide and magnesium stearate were first weighed and mixed thoroughly. Granules were then formed with 15% polyvinylpyrrolidone (PVP) in 25% ethanol as adhesive material and dried under vacuum overnight. The granules were sieved through a 22-mesh screen and weighed based on the single tablet along with exenatide and magnesium stearate. The composition was mixed and pressed into tablets. All tablets were weighed individually, and those tablets with more than 5% deviation from the mean tablet weight were excluded from further experiments.

TABLE 15
Core tablet formulation (amounts shown in milligrams).

| Exenatide | SCA | MCC | CCS | HPMC | PVP | MST | Silica | Total |
|---|---|---|---|---|---|---|---|---|
| 1.5 | 200 | 44.4 | 30 | 9 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose; MST—magnesium stearate; silica—silica powder.

8.2 Bioadhesive Layer

The core tablets were coated with hydroxypropyl methylcellulose (HPMC) with or without enteric material EUDRAGIT® L30D-55. For coating without L30D-55, an aqueous solution of 6% HPMC and 1.5% polyethylene glycol, MW 6000 (PEG 6000) was used in a tablet pan coater (BY300A, Yellow Sea Machinery). The weight gain due to the bioadhesive layer was 3-4 mg. For coating with L30D-55, an aqueous solution of 3% HPMC and 0.6% L30D-55 was used in the tablet pan coater. The coated tablets were dried at 40° C. for about 14 hrs. The weight gain due to the bioadhesive layer was 3 mg.

8.3 Unidirectional Release Layer

The tablets coated with a bioadhesive layer were subsequently coated with a semi-permeable cellulose acetate layer. The tablets were first covered on one side with adhesive paper to create unidirectional release openings. Next, the tablets were coated in a pan coater with a unidirectional coating solution containing 3% cellulose acetate and 1.2% polyethylene glycol, MW 2000 (PEG 2000) in a mixture of acetone and formic acid (9:1 v/v) and dried at 40° C. overnight. The weight gain due to the unidirectional release layer was 2 mg. Finally, the adhesive paper was peeled off to expose the unidirectional release openings.

8.4 Enteric Layer

The tablets coated with a bioadhesive HPMC layer without L30D-55 were further coated with an enteric layer. The coating mixture contained 200 g L30D-55, 12 g ultrafine talc powder, and 6 g polyethylene glycol, MW 6000 (PEG 6000), homogenized in water to a final volume of 400 ml. The coating was performed in a small scale pan coater, and the tablets were dried at 40° C. for 14 hrs. The weight gain due to the enteric layer was 25 mg.

8.5 Absorption of Oral Exenatide in Dogs

The absorption of exenatide in formulations containing 200 mg sodium caprate was evaluated in healthy beagles.

Twelve beagle dogs with body weights between 8-12 kg were housed in an animal facility (Shanghai TCM University Animal Center). Water was supplied ad libitum. The dogs were randomly divided into four groups, and repeated treatments were performed with 1 week washout period. The dogs were fasted overnight, and the tablets were fed directly with 10 ml water. Food was restricted during the study. The treatment groups evaluated in this experiment are summarized in Table 16.

TABLE 16
Treatment groups for evaluating absorption
of oral exenatide in dogs.

| Group | Group | |
|---|---|---|
| # | ID | Treatment |
| 1 | Blank | Placebo tablet |
| 2 | SC | Subcutaneous injection of exenatide, 60 μg/dog |
| | | in 10 mM sodium acetate, pH 4.0 |
| 3 | No enteric | 1.5 mg exenatide and 200 mg sodium caprate in |
| | layer | HPMC/L30D-55 coated tablet, further coated |
| | | with a cellulose acetate layer |
| 4 | Enteric | 1.5 mg exenatide and 200 mg sodium caprate in |
| | layer | HPMC coated tablet, subsequently coated with |
| | | cellulose acetate and L30D-55 enteric layers |

After treatment, 1.5 ml blood samples were drawn from the vein catheter in heparinized tubes at various time points, and 0.5-0.6 ml serum samples were recovered after centrifugation at 3000 rpm for 10 min. The samples were frozen at −20° C., and exenatide concentration was measured using an ELISA kit (Phoenix Pharmaceuticals, Inc.).

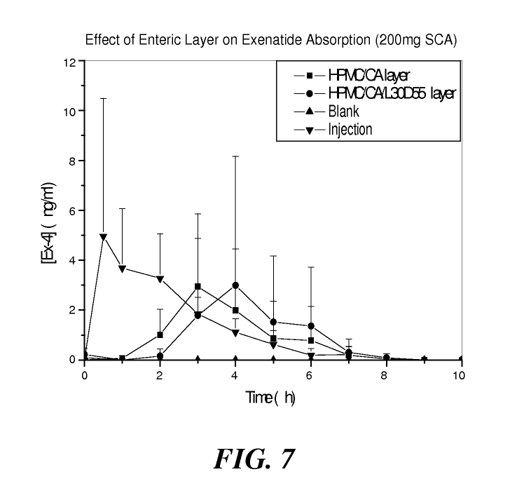

All the samples including standards were adjusted to 20% serum. The serum concentrations of exenatide were calculated based on the standard curve. The kinetics of exenatide absorption for each treatment group is summarized in FIG. 7.

The results indicate that absorption of oral exenatide in the form of HPMC and cellulose acetate coated tablet in the presence of 200 mg sodium caprate is equally effective compared with tablets coated with HPMC, cellulose acetate and enteric material. In the absence of an enteric outer layer, a maximum concentration of exenatide was reached about an hour earlier than in the presence of the enteric layer (3 hrs v. 4 hrs). However, neither the maximum concentration nor the area under the curve seemed to be significantly affected by the presence of the enteric layer. Therefore, while the enteric layer may optionally be used in some embodiments, it does not appear to be a critical component of the present invention.

The early absorption and response demonstrated by the two layered formulation are desirable in clinical settings.

### Example 9

### Effect of Oral Insulin in Somatostatin Infused Dogs

9.1 Preparation of Calcium Phosphate Insulin Nano-Particles

Calcium phosphate insulin particles were prepared and used as drug carrier in this study. Insulin (5 mg/ml) was dissolved in 40 ml solution A containing 20 mM sodium dibasic phosphate, 20 mM HEPES buffer at pH 6.9, 2% PEG (molecular weight 6000), and 0.5% ursodeoxycholate (UDCA). UDCA was dissolved in 1.5 ml ethanol before addition. Equal volume (40 ml) solution B containing 0.01N HCl and 60 mM CaCl₂ was mixed with solution to induce precipitation. The particles were centrifuged at 15000 rpm for 30 min and recovered particles were dried completely under vacuum.

The insulin content in the particles was measured by suspending the particles at 0.2 mg/ml in 50 mM sodium phosphate buffer at pH 9.1 at 37° C. for 15 min. Insulin was estimated by reverse phase high-performance liquid chromatography (RP-HPLC) using a known amount insulin as standard. Insulin content in this particular batch of calcium phosphate particles was 0.56 mg/mg and insulin recovery was 99%.

9.2 Core Tablet Fabrication

The core tablets were fabricated according to the formula listed in Table 16 by compression with a single tablet press. All the components except insulin, silica powder and magnesium stearate were first weighed and mixed thoroughly. Granules were then formed with 8% polyvinylpyrrolidone (PVP) in 25% ethanol as adhesive material and dried under vacuum overnight. The granules were sieved through a 22-mesh screen and weighed based on the single tablet along with insulin, silica powder and magnesium stearate. The composition was mixed and pressed into tablets. All tablets were weighed, and those tablets with more than 5% deviation from the mean tablet weight were excluded from further experiments. Tablet hardness, thickness and friability were also monitored.

TABLE 16
Core tablet formulation (amounts shown in milligrams).

| Insulin | SCA | MCC | CCS | HPMC | PVP | MST | Silica | Total |
|---|---|---|---|---|---|---|---|---|
| 1.8 mg | 200 | 42 | 30 | 9 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose 4000; MST—magnesium stearate; silica—silica powder.

9.3 Bioadhesive Layer

The core tablets were coated with hydroxypropyl methylcellulose (HPMC E50) and enteric material EUDRAGIT® L30D-55. An aqueous suspension of 3% HPMC and 0.6% L30D-55 was used in the tablet pan coater (BY300A, Yellow Sea Machinery). The coated tablets were dried at 40° C. for about 14 hrs. The weight gain due to the bioadhesive layer was 3 mg.

9.4 Unidirectional Release Layer

The tablets coated with a bioadhesive layer were subsequently coated with a semi-permeable cellulose acetate layer. The tablets with diameter of 9 mm were first covered on one side with adhesive paper having circular shape of 7 mm diameter. Next, the tablets were coated in a pan coater with a unidirectional coating solution containing 3% cellulose acetate and 1.2% polyethylene glycol, MW 2000 (PEG 2000) in a mixture of acetone and formic acid (9:1 v/v) and dried at 40° C. overnight. The weight gain due to the unidirectional release layer was 2 mg. Finally, the circular sticker was peeled off to expose the unidirectional release opening.

9.5 Insulin Release Profile

Insulin release was evaluated first in 100 ml 0.01N HCl for 2 hrs in a dissolution apparatus with a basket design at 100 rpm agitation and 37° C. Aliquots were taken at 1 hr and 2 hrs and insulin content was measured by RP-HPLC. The tablets were then transferred into 100 ml of simulated intestinal fluid (SIF) at pH 6.8 under same conditions, and aliquots were subject to RP-HPLC at various time points to measure insulin content.

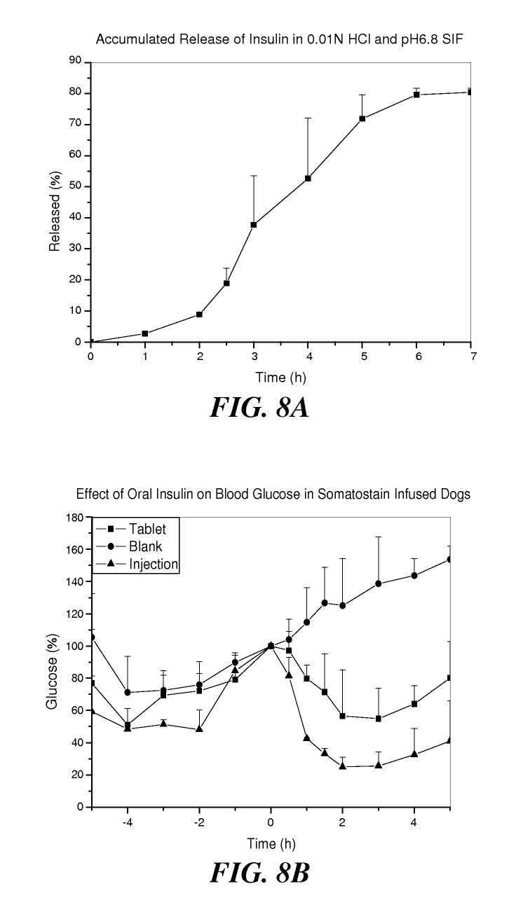

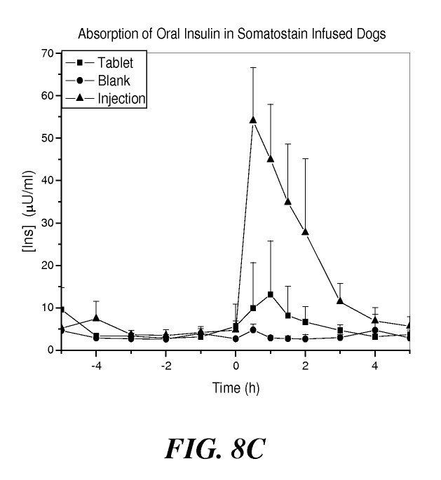

The insulin release profile is shown in FIG. 8A. The tablets were stable in the acid solution, as evidenced by the observation that less than 10% of insulin was released at 2 hr incubation. Insulin release in SIF was gradual and completed in about 3-4 hrs.

9.6 Effect of Oral Insulin in Somatostatin Infused Dogs

The absorption of oral insulin was evaluated in beagles under somatostatin infusion at 1 μg/kg/min via an indwelling vein catheter. Infusion of somatostatin suppresses endogenous glucagon and insulin secretion (Sakurai et al., J. Clin. Invest. 54:1395, 1974). The blood glucose level in these animals initially decreases, followed by a rapid increase due to insulin suppression. This model allows simultaneous pharmacokinetic and pharmacodynamic evaluation of oral insulin.

Twelve beagle dogs with body weights between 8-12 kg were housed in an animal facility. The dogs were randomly divided into three groups and fasted overnight. Water was supplied ad libitum. Somatostatin infusion (1 μg/kg/min) was performed using a balloon infusion pump via an indwelling vein catheter, and tablets were fed with 10 ml water at 4 hr after initiation of somatostatin infusion. Food was restricted during the study, and blood samples were collected at various time points in heparinized tubes. The treatment groups evaluated in this experiment are summarized in Table 17.

TABLE 17
Treatment groups for oral insulin administration
in somatostatin infused dogs.

| Group ID | N | Treatment |
|---|---|---|
| Blank | 3 | Placebo tablet |
| SC | 3 | Subcutaneous injection of insulin, 3.5 U/dog in |
| | | 0.01M HCl |
| FTP | 3 | 50 U oral insulin tablet |

Blood glucose concentration was measured with a glucometer and matching testing strips (Johnson & Johnson, OneTouch®), and serum samples of 0.5-0.6 ml were recovered after blood samples were collected and centrifuged at 3000 rpm for 10 min. The samples were frozen at −20° C., and insulin concentration was measured by ELISA (Linco).

The effects of oral insulin treatment on blood glucose and serum insulin concentrations are shown in FIGS. 8B and 8C, respectively.

As shown in FIG. 8B, the blood glucose concentration initially decreased, followed by a substantial increase in blank tablet treated animals, as expected. Insulin injection induced a rapid and profound reduction of glucose levels. Similarly, treatment with oral insulin also induced a significant reduction of blood glucose. Estimated bio-potency was 5% based on the AUC of glucose levels.

As shown in FIG. 8C, the insulin levels were suppressed during the entire experiment in blank tablet treated animals, while insulin injection resulted in a large increase in insulin concentration. Interestingly, there was only a moderate increase in insulin concentration in animals treated with oral insulin. Bioavailability in this study was about 2%, or about 40% of bio-potency. This observation confirms that oral insulin, much like portal or natural insulin secretion, results in a higher bio-potency and lower peripheral insulin. Therefore, the probability of hypoglycemia, a critical problem limiting clinical use of insulin, is significantly reduced.

### Example 10

### Effect of Two Layered Oral Insulin Tablet in Somatostatin Infused Dogs

10.1 Core Tablet Fabrication

The core tablets were fabricated according to the formula listed in Table 18 by compression with a single tablet press. All the components except insulin, silica powder and magnesium stearate were first weighed and mixed thoroughly. Granules were then formed with 8% polyvinylpyrrolidone (PVP) in 25% ethanol as adhesive material and dried under vacuum overnight. The granules were sieved through a 22-mesh screen and the amount needed for each tablet was weighed individually and followed by addition of insulin, silica powder and magnesium stearate. The composition was mixed and pressed into tablets. Hardness, friability, and thickness of the tablets were also measured.

TABLE 18
Core tablet formulation (amounts shown in milligrams).

| Insulin | SCA | MCC | CCS | HPMC | PVP | MST | Silica | Total |
|---|---|---|---|---|---|---|---|---|
| 1.8 | 200 | 42 | 30 | 9 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose; MST—magnesium stearate; silica—silica powder.

10.2 Bioadhesive Layer

The core tablets were coated with hydroxypropyl methylcellulose (HPMC) with or without enteric material EUDRAGIT® L30D-55. For coating without L30D-55, an aqueous solution of 6% HPMC and 1.5% polyethylene glycol, MW 6000 (PEG 6000) was used in a tablet pan coater (BY300A, Yellow Sea Machinery). The weight gain due to the bioadhesive layer was 3-4 mg. For coating with L30D-55, an aqueous solution of 3% HPMC and 0.6% L30D-55 was used in the tablet pan coater. The coated tablets were dried at 40° C. for about 14 hrs. The weight gain due to the bioadhesive layer was 3 mg.

10.3 Unidirectional Release Layer

The tablets coated with a bioadhesive layer were subsequently coated with a semi-permeable cellulose acetate layer. The 10 mm diameter tablets were first covered on one side with circular stickers of 7 mm diameter. Next, the tablets were coated in a pan coater with a unidirectional coating solution containing 3% cellulose acetate and 1.2% polyethylene glycol, MW 2000 (PEG 2000) in a mixture of acetone and formic acid (9:1 v/v). The tablets were dried at 40° C. overnight after coating was completed. The weight gain due to the unidirectional release layer was 2 mg. Finally, the adhesive paper was peeled off to expose the unidirectional release openings.

10.4 Enteric Layer

The tablets coated with a bioadhesive HPMC layer without L30D-55 were further coated with an enteric layer. The coating mixture contained 200 g L30D-55, 12 g ultrafine talc powder, and 6 g polyethylene glycol, MW 6000 (PEG 6000), homogenized in water to a final volume of 400 ml. The coating was performed in a small scale pan coater, and the tablets were dried at 40° C. for 14 hrs. The weight gain due to the enteric layer was 25 mg.

10.5 Effect of Oral Insulin on Glucose

The effects of oral insulin with the two-layer formulation (core tablet coated with bioadhesive layer with L30D55 followed by cellulose acetate unidirectional coating) and three-layer formulation (core tablet coated with bioadhesive layer without L30D55 followed by cellulose acetate coating and subsequent enteric coating) were evaluated in beagle dogs infused with somatostatin as described in Example 9.

Twelve beagle dogs with body weights between 8-12 kg were housed in an animal facility. Water was supplied ad libitum. The dogs were randomly divided into four groups and fasted overnight. The tablets were fed directly into the throat with 10 ml water about 4 hrs after initiation of somatostatin infusion. Food was restricted during the study. The treatment groups evaluated in this experiment are summarized in Table 19.

TABLE 19
Treatment groups for oral insulin formulation.

| Group ID | N | Treatment |
|---|---|---|
| Blank | 3 | Placebo tablet |
| SC | 3 | Subcutaneous injection of insulin, 3.5 U/dog in |
| | | 0.01N HCl |
| No enteric layer | 3 | 50 U two layer insulin tablets |
| Enteric layer | 4 | 50 U three layer insulin tablets |

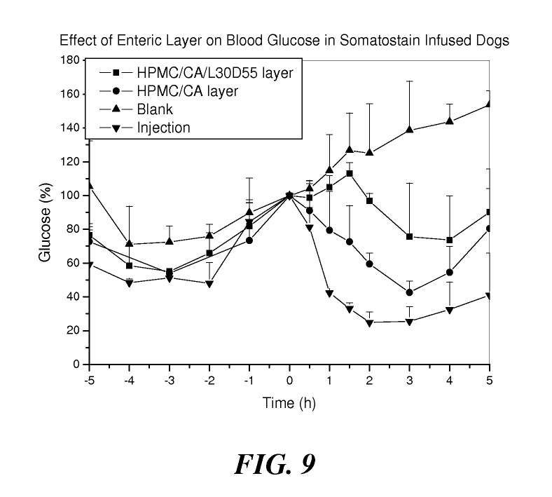

Blood samples were collected and blood glucose concentration was measured with a glucometer. The effects of the two oral insulin formulations on blood glucose are shown in FIG. 9.

As shown in FIG. 9, the oral insulin tablet administration induced a significant blood glucose reduction, similar to insulin injection, whereas blood glucose in blank treated animals showed an initial decrease followed by rapid increase. In the absence of the enteric outer layer, the blood glucose decrease was first observed at 1 hr after tablet administration, while the same effect was only seen at 4 hr after the enteric coated tablet administration.

This result is consistent with the observation in the similar formulations with exenatide and demonstrates that early response by the two-layer formulation is desirable in clinical settings.

### Example 11

### Direct Compressed Tablets

Direct compression without granulation is a simple and economical manufacturing process for tablets, which tends to maintain drug crystal structure and stability. A major challenge for low dose tablets manufactured using a direct compression process is to achieve acceptable uniformity of content.

Formulations of insulin and exenatide listed in Table 20 were produced by direct compression. Insulin and exenatide powders were sieved through a 200-mesh screen and mixed thoroughly with all the excipients. The compositions were compressed into tablets using a rotary press. Hardness, friability, and thickness of the tablets were also measured.

TABLE 20
Core tablet formulation (amounts shown in milligrams).

| Insulin or Exenatide | SCA | MCC | HPMC | MST | Silica | Total |
|---|---|---|---|---|---|---|
| 1 | 100 | 95 | 16 | 1 | 6.6 | 200 |

SCA—sodium caprate; MCC—microcellulose crystalline; CCS—croscarmellose sodium; HPMC—hydroxypropyl methylcellulose; MST—magnesium stearate; silica—silica powder.

Blend uniformities of insulin and exenatide were evaluated according to U.S. Pharmacopeia General Chapter <905>, “Uniformity of Dosage Units” (“USP <905>”), and samples of the same weight (equal to that of a tablet) were collected from different locations after the blends were mixed completely. Insulin or exenatide content was measured by RP-HPLC. The blend uniformity results are shown in the Table 21.

TABLE 21
Uniformity of Direct Compression Blends

| Batch | Drug | Content/Tablet Weight | Mean | RSD |
|---|---|---|---|---|
| 178-2 | Exenatide | 600 μg/200 mg | 672 ± 83 | 12.4% |
| 176 | Insulin | 1 mg(27 U)/200 mg | 26 ± 0.7 | 2.8% |

The results indicate that the blend uniformity of insulin at unit dose 1 mg (0.5% of tablet content) is acceptable. In contrast, exenatide at 0.6 mg at 0.27% content failed the uniformity test. Exenatide powder exhibits higher hydroscopic activity and may contribute to this difficulty. Therefore, the direct compression process for fabricating tablets is feasible, and drugs with different properties such as hydroscopy and particle size may present various levels of difficulty in achieving acceptable uniformity.

### Example 12

### Non-Solvent Granulation in Tablet Production

As noted above, direct compression presents a challenge in terms of content uniformity for low-dose drugs like exenatide, whereas wet granulation is detrimental to drug stability. Therefore, fabrications of insulin and exenatide tablets were prepared by non-solvent granulation. The compositions for different lots are listed in Tables 22-26.

Insulin and exenatide powders were sieved through a 200-mesh screen and suspended in 8% PVP in ethanol. The drug suspensions were sonicated lightly until no visible large particles were detected. All other excipients except silica powder and magnesium stearate were weighed and pre-mixed thoroughly in a granulator. The drug suspensions were added to the excipient blends to form granules, which then were sieved through a 18-mesh screen and dried under vacuum. After drying and addition of silica powder and magnesium stearate, the compositions were compressed into tablets using a rotary press. Hardness, friability, and thickness of the tablets were also measured.

TABLE 22
Core tablet formulation for insulin at 1 mg
(amounts shown in milligrams; Lot 181).

| Insulin | SCA | MCC | CCS | HPMC | PVP | MST | Silica | Total |
|---|---|---|---|---|---|---|---|---|
| 1 | 200 | 55 | 15 | 15 | 10 | 1.5 | 3 | 300 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder.

TABLE 23
Core tablet formulation for insulin at 0.8
mg (amounts shown in milligrams; Lot 182).

| Insulin | SCA | MCC | CCS | HPMC | PVP | MST | Silica | Total |
|---|---|---|---|---|---|---|---|---|
| 0.8 | 200 | 55 | 15 | 15 | 10 | 1.5 | 3 | 300 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder.

TABLE 24
Core tablet formulation for exenatide at 0.6
mg (amounts shown in milligrams; Lot 178-1).

| Exenatide | SCA | MCC | HPMC | PEG | MST | Silica | Total |
|---|---|---|---|---|---|---|---|
| 0.6 | 100 | 95 | 16 | 40 | 1 | 2 | 250 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder.

TABLE 25
Core tablet formulation for exenatide at 0.4
mg (amounts shown in milligrams; Lot 183).

| Exenatide | SCA | MCC | CCS | HPMC | PVP | MST | Silica | Total |
|---|---|---|---|---|---|---|---|---|
| 0.4 | 100 | 155 | 15 | 15 | 10 | 1.5 | 3 | 300 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder.

TABLE 26
Core tablet formulation for exenatide at 0.4
mg (amounts shown in milligrams; Lot 184).

| Exenatide | SCA | MAN | MCC | CCS | HPMC | PVP | MST | Silica | Total |
|---|---|---|---|---|---|---|---|---|---|
| 0.4 | 100 | 95 | 60 | 15 | 15 | 10 | 1.5 | 3 | 300 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder; MAN—mannitol.

Insulin or exenatide content in these tablets are measured by RP-HPLC and content uniformity is evaluated according to USP <905>. The results are shown in Table 27.

TABLE 27
Insulin and exenatide content uniformity in Lots 178-184.

| Lot | Drug | Content/Tablet Weight | Mean | RSD |
|---|---|---|---|---|
| 178-1 | Exenatide | 600 μg/220 mg | 583 ± 6 | 1.0% |
| 181 | Insulin | 1 mg(27 U)/300 mg | 28 ± 0.2 | 0.8% |
| 182 | Insulin | 0.8 mg(22 U)/300 mg | 22 ± 0.3 | 1.2% |
| 183 | Exenatide | 400 μg/300 mg | 424 ± 14 | 3.3% |
| 184 | Exenatide | 400 μg/300 mg | | |

These results demonstrate that the content uniformities of insulin and exenatide tablets fabricated using non-solvent granulation are acceptable.

### Example 13

### Stability of Exenatide in Different Formulation & Processes

Stability of exenatide in various formulations was evaluated under stress conditions. The formulations tested are shown in Table 28.

TABLE 28
Stability of exenatide.

| Formulation | Exenatide | Process |
|---|---|---|
| Exenatide powder | 1 mg | N/A |
| Direct blend with all excipients | 1 mg | N/A |
| Tablet with all excipients | 1 mg | Wet granulation |
| Tablet with all excipients | 1 mg | Direct compression |
| Tablet with all excipients | 1 mg | Non-solvent granulation |
| Tablet with all excipients plus | 1 mg | Wet granulation |
| gelatin or mannitol | | |
| Tablet with all excipients plus | 1 mg | Non-solvent granulation |
| PEG | | |

Excipients contained in the basic formulation are listed in Table 29.

TABLE 29
Basic core tablet formulation (amounts shown in milligrams).

| Exenatide | SCA | MCC | CCS | HPMC | PVP | MST | Silica | Total |
|---|---|---|---|---|---|---|---|---|
| 1 | 200 | 42 | 30 | 9 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose 4000; MST—magnesium stearate; silica—silica powder.

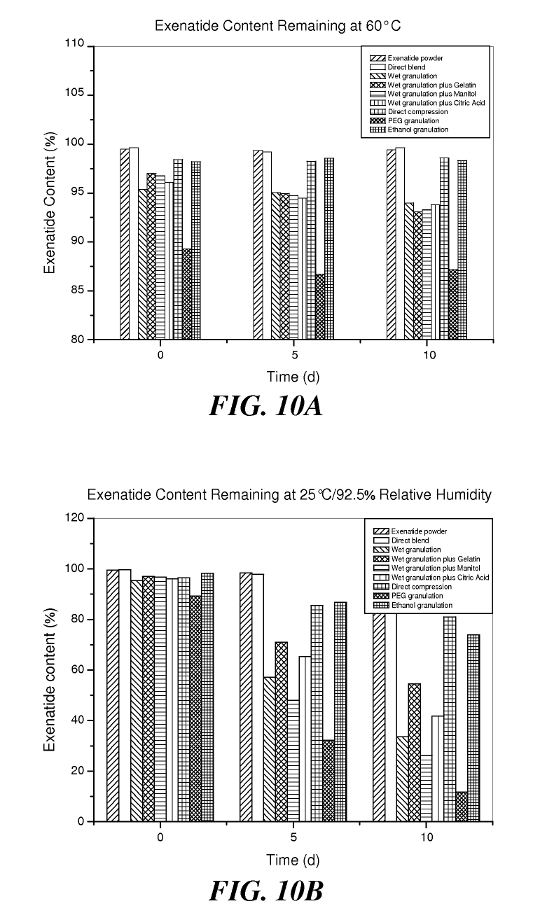

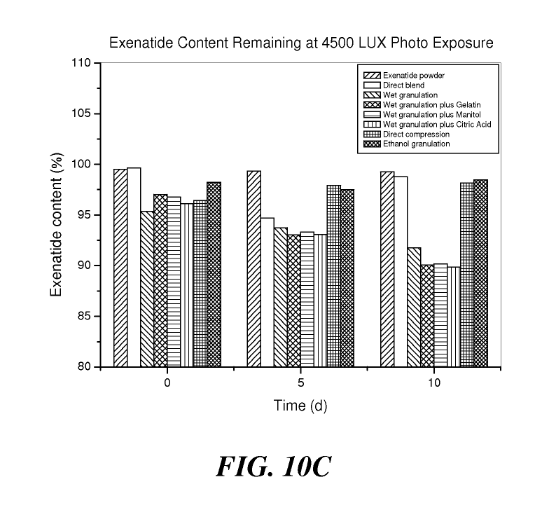

All formulations were placed in a Petri dish and placed under stress conditions, including 60° C. temperature, 25° C. at 92.5% relative humidity, and 4500 lux photo exposure, for 10 consecutive days. Exenatide content in each formulation was measured by RP-HPLC at 0, 5, and 10 days. The content remaining in each formulation is shown in FIGS. 10A-10C.

The results demonstrate that exenatide powder is relatively stable at stress conditions, and exenatide content remains at 95% after 10 day exposure at high temperature, high humidity and photo exposure. The direct blend with all the excipients shows a similar stability profile, suggesting that the excipients are compatible with exenatide in the tablet formulation.

However, tablets fabricated by wet granulation show relatively unstable behavior, and severe loss of exenatide is observed at 5 and 10 day exposure. Addition of gelatin or mannitol did not alleviate the instability. In fact, the instability of exenatide was aggravated, particularly at high humidity. This observation may be attributed to the ability of gelatin and mannitol to absorb a significant amount of moisture at high humidity.

In contrast, tablets fabricated by direct compression or non-solvent granulation show good stability at these conditions, similar to exenatide powder. It is notable that addition of low molecular weight PEG caused instability even in formulations produced by the non-solvent granulation process, which may be attributed to partial solubility of exenatide in low molecular weight PEG.

### Example 14

### Effect of Oral Insulin in Dogs

14.1 Core Tablet Fabrication

The core tablets were fabricated according to the formula listed in Table 30 by non-solvent granulation followed by compression with a rotary press. All the components except insulin, PVP, silica powder, and magnesium stearate were first weighed and mixed thoroughly in a granulator. Insulin powder was suspended in 8% polyvinylpyrrolidone (PVP) in ethanol and sonicated to breakdown the large insulin particles. Granules were formed by adding the insulin suspension to the other components and drying the mixture under vacuum overnight. Silica powder and magnesium stearate were added to the dried granules and the composition was mixed and pressed into tablets. Hardness, friability, and thickness of the tablets were also measured.

TABLE 30
Core tablet formulation (amounts shown in milligrams).

| Insulin | SCA | MCC | CCS | HPMC | PVP | MST | Silica | Total |
|---|---|---|---|---|---|---|---|---|
| 0.9 | 100 | 42 | 15 | 15 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose; MST—magnesium stearate; silica—silica powder.

14.2 Bioadhesive Layer

The core tablets were coated with hydroxypropyl methylcellulose (HPMC) with 6% HPMC and 1.5% polyethylene glycol (MW 6000) solution in a tablet pan coater (BY300A, Yellow Sea Machinery). The weight gain due to the bioadhesive layer was 4 mg.

14.3 Unidirectional Release Layer

The tablets coated with the bioadhesive layer were subsequently coated with a semi-permeable ethyl cellulose layer. The 10 mm diameter tablets were first covered on one side with circular stickers of 7 mm diameter. Next, the tablets were coated in a pan coater with a unidirectional coating solution containing 3% ethyl cellulose and 1.2% polyethylene glycol (MW 2000) in ethanol. The tablets were dried at room temperature after coating was completed. The weight gain due to the unidirectional release layer was 2 mg. Finally, the adhesive paper was peeled off to expose the unidirectional release openings.

14.4 Enteric Layer

The tablets were further coated with an enteric layer. The coating mixture contained 8% L30D-55, 2% polyethylene glycol (MW 6000) in ethanol. The coating was performed in a small scale pan coater and the weight gain due to the enteric layer was 20 mg.

14.5 Effect of Oral Insulin on Blood Glucose Levels in Normal Dogs

The effect of oral insulin administration on blood glucose levels was evaluated in normal beagle dogs.

Nine beagle dogs with body weights between 8-12 kg were housed in an animal facility. Water was supplied ad libitum. The dogs were randomly divided into three groups and fasted overnight. The tablets were fed directly into the throat with 10 ml water. Food was restricted during the study. The treatment groups evaluated in this experiment are summarized in Table 31.

TABLE 31
Treatment Groups for Oral Insulin Formulation

| Group ID | N | Treatment |
|---|---|---|
| Blank | 3 | Placebo tablet |
| SC | 3 | Subcutaneous injection of insulin, 3.5 U/dog in |
| | | 0.01N HCl |
| 50 U | 3 | 2 × 25 U oral insulin tablets |

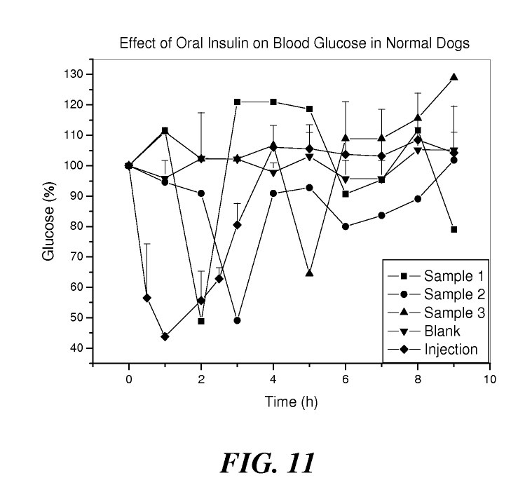

Blood samples were collected and blood glucose concentration was measured with a glucometer. The effect of the oral insulin administration on blood glucose is shown in FIG. 11.

As shown in FIG. 11, oral insulin tablets induced significant blood glucose reduction within 2 to 3 hours after administration, similar to insulin injection, whereas blood glucose in blank treated animals was relatively steady.

The blood glucose is often controlled within a tight range in normal subjects. Therefore, the glucose reduction even in sc injection was transit. Oral insulin tablet induced glucose reduction in similar magnitude and transit fashion as injected insulin in normal beagles.

### Example 15

### Laser Ablation Process in Tablet Production

15.1 Core Tablet Fabrication

The core tablets were fabricated according to the formula listed in Table 32 by compression with a rotary press. All the components except exenatide, silica powder and magnesium stearate were first weighed and mixed thoroughly. Granules were then formed with exenatide dissolved in 8% polyvinylpyrrolidone (PVP) in 25% ethanol as adhesive material, and the granules were dried under vacuum overnight, sieved through a 22-mesh screen, and silica powder and magnesium stearate were added. The composition was mixed and pressed into tablets. Tablet weight, hardness, thickness and friability were monitored.

TABLE 32
Core tablet formulation (amounts shown in milligrams).

| Exenatide | SCA | MCC | CCS | HPMC | PVP | MST | Silica | Total |
|---|---|---|---|---|---|---|---|---|
| 1 | 200 | 42 | 30 | 9 | 10 | 1.5 | 3 | 300 |

SCA—sodium caprate; MCC—microcellulose crystalline (Avicel PH-101); CCS—croscarmellose sodium; PVP—polyvinyl pyrrolidone; HPMC—hydroxypropyl methylcellulose 4000; MST—magnesium stearate; silica—silica powder.

15.2 Bioadhesive Layer

The core tablets were coated with hydroxypropyl methylcellulose (HPMC E50) and enteric material EUDRAGIT® L30D-55 using an aqueous suspension of 3% HPMC and 0.6% L30D-55 in a pan coater (BY300A, Yellow Sea Machinery). The coated tablets were dried at 40° C. for about 14 hrs. The weight gain due to the bioadhesive layer was 3 mg.

15.3 Unidirectional Release Layer

The above tablets were further coated with a semi-permeable cellulose acetate layer. For manual process, 9 mm tablets were first covered on one side with circles of adhesive paper of 7 mm diameter, followed by coating with a solution containing 3% cellulose acetate and 1.2% polyethylene glycol, MW 2000 (PEG 2000) in a mixture of acetone and formic acid (9:1 v/v) in a pan coater. The circular stickers were peeled off to expose the unidirectional release openings after coating and drying were completed.

For laser ablation process, the tablets were not covered with stickers and coated with the same cellulose acetate solution. The coated tablets were dried at 40° C. overnight. The completed tablets were ablated with a laser drilling equipment (CMS) to form a 7 mm diameter aperture similar to the one obtained by the manual process described above. The integrity of bioadhesive coating was verified by acid susceptibility test in simulated gastric fluid for 2 hrs.

The weight gain due to the unidirectional release layer was 2-3 mg in both cases.

15.4 Absorption of Oral Exenatide in Normal Dogs

The absorption of oral exenatide mediated by the unidirectional release coatings formed by the manual and laser ablation processes was evaluated in normal beagles.

Twelve beagle dogs with body weights between 8-12 kg were housed in an animal facility. The dogs were randomly divided into four groups and fasted overnight. Water was supplied ad libitum. Tablets were fed directly into the throat with 10 ml water and food was restricted during the study. Blood samples were collected at various time points in heparinized tubes. The treatment groups evaluated in this experiment are summarized in Table 33.

TABLE 34
Treatment groups for oral exenatide.

| Group ID | N | Treatment |
|---|---|---|
| Blank | 3 | Placebo tablet |
| SC | 3 | Subcutaneous injection of exenatide, 60 μg/dog in |
| | | 10 mM sodium acetate, pH 4.5 |
| Sticker | 3 | 1 mg oral exenatide tablet with manual process |
| Laser | 3 | 1 mg oral exenatide tablet with laser process |

Serum samples of 0.5-0.6 ml were recovered after blood samples were collected and centrifuged at 3000 rpm for 10 min. The samples were frozen at −20° C., and exenatide concentration was measured using an ELISA kit (Phoenix).

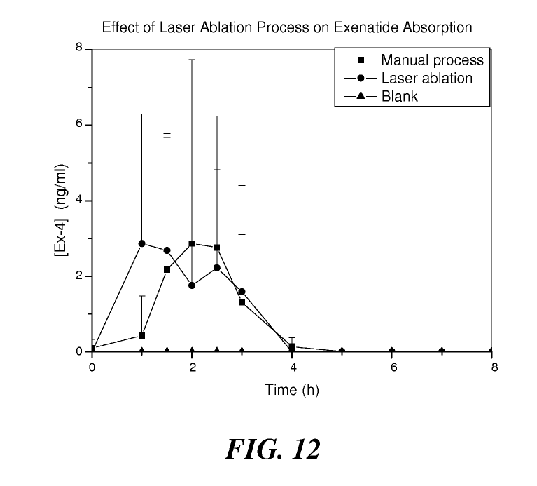

The effect of oral exenatide treatment on serum exenatide concentration is shown in FIG. 12. The formulations fabricated by the manual or laser ablation process to form the unidirectional release opening achieved similar extent of absorption, although it appears that the formulation fabricated using laser ablation had somewhat faster absorption kinetics.

### Example 16

### Dose Response of Oral Insulin in Somatostatin Infused Dogs

16.1 Core Tablet Fabrication

The core tablets were fabricated according to the formula listed in Table 34 by compression with a single tablet press. All the components except insulin, silica powder and magnesium stearate were first weighed and mixed thoroughly. Granules were then formed with 8% polyvinylpyrrolidone (PVP) in 25% ethanol as adhesive material and dried under vacuum overnight. The granules were sieved through a 22-mesh screen, and the amount needed for each tablet was weighed individually, followed by the addition of insulin, silica powder and magnesium stearate. The composition was mixed and pressed into tablets. Hardness, friability, and thickness of the tablets were also measured.

TABLE 34
Core tablet formulation (amounts shown in milligrams).

| Insulin | SCA | MCC | CCS | HPMC | PVP | MST | Silica | Total |
|---|---|---|---|---|---|---|---|---|
| 0.9 or | 200 | 42 | 30 | 9 | 10 | 1.5 | 3 | 300 |
| 1.8 mg | | | | | | | | |

SCA—sodium caprate; MCC—microcellulose crystalline (Avicel PH-101); CCS—croscarmellose sodium; PVP—polyvinyl pyrrolidone; HPMC—hydroxypropyl methylcellulose; MST—magnesium stearate; silica—silica powder.

16.2 Bioadhesive Layer

The core tablets were coated with an aqueous suspension of 3% HPMC and 0.6% L30D-55 in a tablet pan coater. The coated tablets were dried at 40° C. for 14 hrs. The weight gain due to the bioadhesive layer was 3 mg.

16.3 Unidirectional Release Layer

The tablets coated with the bioadhesive layer were subsequently coated with a semi-permeable cellulose acetate layer. The 10 mm diameter tablets were first covered on one side with circular sticker of 7 mm diameter. Next, the tablets were coated in a pan coater with a unidirectional coating solution containing 3% cellulose acetate and 1.2% polyethylene glycol, MW 2000 (PEG 2000) in a mixture of acetone and formic acid (9:1 v/v). The tablets were dried at 40° C. overnight after coating completed. The weight gain due to the unidirectional release layer was 2 mg. Finally, the adhesive paper was peeled off to expose the unidirectional release openings.

16.4 Effect of Oral Insulin on Blood Glucose Levels in Somatostatin Treated Dogs

The effect of oral insulin was evaluated in beagle dogs infused with somatostatin, as described in Example 9.

Twelve beagle dogs with body weights between 8-12 kg were housed in an animal facility. Water was supplied ad libitum. The dogs were randomly divided into five groups and fasted overnight. The tablets were fed directly into the throat with 10 ml water 4 hrs after initiation of somatostatin infusion. Food was restricted during the study. The treatment groups evaluated in this experiment are summarized in Table 35.

TABLE 35
Treatment groups for oral insulin formulation.

| Group ID | N | Treatment |
|---|---|---|
| Blank | 3 | Placebo tablet |
| SC | 3 | Subcutaneous injection of insulin, 3.5 U/dog in |
| | | 0.01N HCl |
| 25 U | 3 | One 25 U insulin tablets |
| 25 U × 2 | 4 | Two 25 U insulin tablets |
| 50 U | 3 | One 50 U insulin tablets |

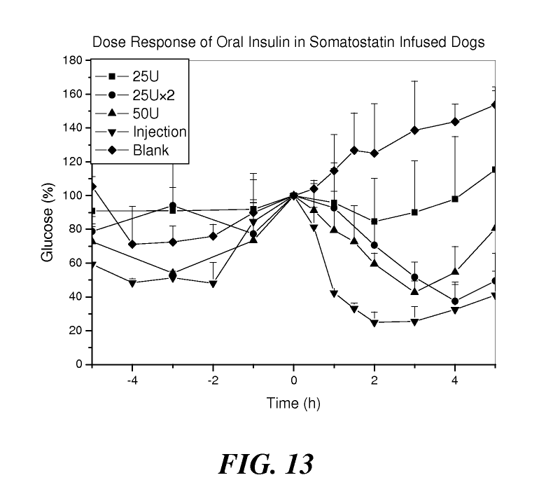

Blood samples were collected and blood glucose concentration was measured with a glucometer. The effects of two oral insulin formulations on blood glucose are shown in FIG. 13.

As shown in FIG. 13, oral insulin tablets induced a significant blood glucose reduction, similar to insulin injection. The glucose reduction was dose-dependent when the response in 25U oral insulin was compared to either 50U oral insulin or 25U×2 treatment. The glucose reduction appeared to be approximately equivalent in the 50U and 25U×2 oral insulin treated groups.

### Example 17

### Effect of Bioadhesive Polymer of Exenatide Release and Absorption

17.1 Core Tablet Fabrication

Exenatide tablets were fabricated using the ethanol granulation process as described earlier. The formulation for this batch of tablets is listed in Table 36. Exenatide power was suspended in 8% PVP in ethanol and sonicated lightly until no visible large particles detected. All other excipients except silica powder and magnesium stearate were weighed and pre-mixed thoroughly in a granulator. The drug suspension was added to the excipient blend to form granules, which were then sieved through 18-mesh screen and dried under vacuum. After the addition of silica powder and magnesium stearate, the composition was compressed into tablets on a rotary press. Hardness, friability, and thickness of the tablets were also measured.

TABLE 36
Core tablet formulation (amounts shown in milligrams; Lot 184).

| Exenatide | SCA | MCC | MAN | CCS | HPMC | PVP | MST | Silica | Total |
|---|---|---|---|---|---|---|---|---|---|
| 0.4 | 100 | 60 | 95 | 15 | 15 | 10 | 1.5 | 3 | 300 |

SCA—sodium caprate; MCC—microcellulose crystalline; CCS—croscarmellose sodium; HPMC—hydroxypropyl methylcellulose; PVP—Polyvinylpyrrolidone; MST—magnesium stearate; silica—silica powder; man—mannitol.

17.2 Bioadhesive Layer

The core tablets were coated with an aqueous suspension of 3% HPMC and 0.6% L30D-55 in 50% ethanol in a tablet pan coater. Alternatively, the core tablets were coated with 2.6% HPMC, 0.8% L30-D55, and 0.6% PEG in 50% ethanol. The coated tablets were dried at 40° C. for 14 hrs. The weight gain due to the bioadhesive layer was 3-4 mg.

17.3 Unidirectional Release Layer

The tablets coated with the bioadhesive layer were subsequently coated with a semi-permeable ethyl cellulose in a pan coater with a unidirectional coating solution containing 3.2% ethyl cellulose, 0.6% polyethylene glycol, MW 2000 and 0.2% triacetin in 85% ethanol. The tablets were dried at 40° C. overnight after coating was completed. The weight gain due to the unidirectional release layer was 2 mg. One side of the tablets was ablated with a laser to form a unidirectional release opening of 7 mm in diameter on these 9 mm diameter tablets.

17.4 Absorption of Exenatide in Normal Dogs

The absorption of exenatide was evaluated in normal beagle dogs.

Twelve beagle dogs with body weights between 8-12 kg were housed in an animal facility. Water was supplied ad libitum. The dogs were randomly divided into four groups and fasted overnight. The tablets were fed directly into the throat with 10 ml water. Food was restricted during the study. The treatment groups evaluated in this experiment are summarized in Table 37.

TABLE 37
Treatment groups for oral insulin formulation.

| Group ID | N | Treatment |
|---|---|---|
| Blank | 3 | Placebo tablet |
| SC | 3 | Subcutaneous injection of insulin, 3.5 U/dog in |
| | | 0.01N HCl |
| Ex-H | 3 | Two 0.4 mg exenatide tablets with 65% HPMC coating |
| Ex-P | 3 | Two 0.4 mg exenatide tablets with 80% HPMC coating |

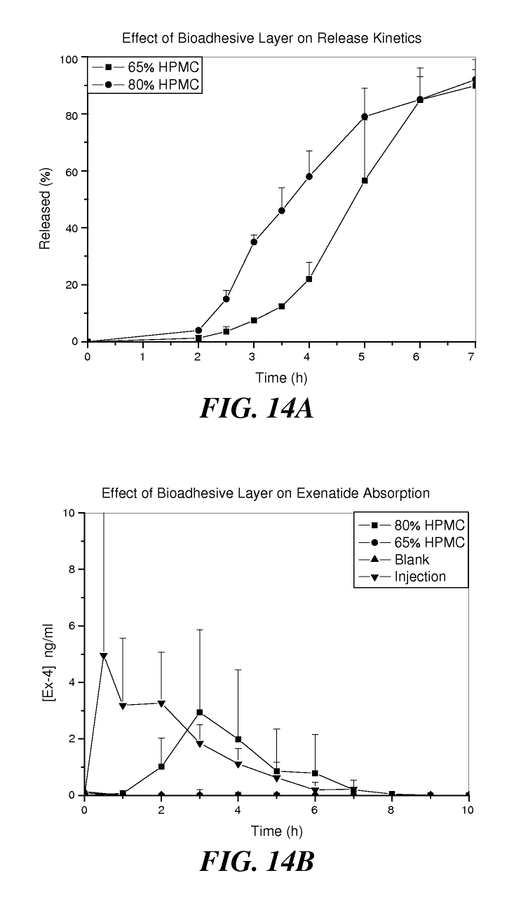

Blood samples were collected at various time points and serum sample were recovered after centrifugation at 3000 rpm for 5 min Serum exenatide concentration was measured using an ELISA assay (Phoenix). The results are shown in FIGS. 14A and 14B.

The results show that the presence of the bioadhesive layer is required and the content of the bioadhesive polymer in the bioadhesive layer has a significant effect on the release and absorption of exenatide.

The preceding examples are included for illustrative purposes and are not intended to limit the scope of the present invention. It is further recognized that various embodiments can be made without departing from the spirit and scope of the present invention by those skilled in the art in light of the present disclosure. It is therefore understood that the present invention embraces all equivalents herein.

1. A pharmaceutical composition comprising:
a) a solid dosage form comprising an effective amount of a therapeutic agent, a permeation enhancer and a pharmaceutically acceptable excipient; and
b) a bioadhesive layer comprising a bioadhesive polymer.

2. The pharmaceutical composition of claim 1, wherein the content of the bioadhesive polymer in the bioadhesive layer ranges between about 50-100%, about 70-90%, or about 80-90% by weight.

3. The pharmaceutical composition of claim 1, wherein the content of the bioadhesive layer in the pharmaceutical composition ranges between about 0.5-10%, about 1-5%, or about 2-3% by weight.

4. The pharmaceutical composition of claim 1, further comprising an impermeable or semi-permeable layer having an opening capable of directing a substantially unidirectional release of the therapeutic agent and the permeation enhancer from the solid dosage form.

5. The pharmaceutical composition of claim 4, wherein the area of the opening covers about 20-90%, about 40-80%, or about 50-70% of the total area of one side of the solid dosage form.

6. The pharmaceutical composition of claim 4, where the content of the impermeable or semi-permeable layer in the pharmaceutical composition ranges between about 0.5-10%, about 1-5%, or about 2-4% by weight.

7. The pharmaceutical composition of claim 1, wherein the therapeutic agent and the permeation enhancer have substantially equivalent relative rates of release from the solid dosage form.

8. The pharmaceutical composition of claim 1, further comprising an enteric layer.

9. The pharmaceutical composition of claim 1, wherein the bioadhesive polymer is selected from a carbomer, a polycarbophil, a hydroxypropyl methylcellulose, a chitosan, and salts and derivatives thereof.

10. The pharmaceutical composition of claim 1, wherein the bioadhesive layer further comprises an enteric polymer.

11. The pharmaceutical composition of claim 4, wherein the impermeable or semi-permeable layer comprises a water impermeable or semi-permeable material.

12. The pharmaceutical composition of claim 11, wherein the water impermeable or semi-permeable material is selected from ethyl cellulose, cellulose acetate, and salts and derivatives thereof.

13. The pharmaceutical composition of claim 11, wherein the impermeable or semi-permeable layer further comprises a plasticizer.

14. The pharmaceutical composition of claim 1, wherein the permeation enhancer is selected from the group consisting of a fatty acid, a medium chain glyceride, a surfactant, a steroidal detergent, an acyl carnitine, an alkanoyl choline, an N-acetylated amino acid, esters, salts and derivatives thereof, and any combination thereof.

15. The pharmaceutical composition of claim 14, wherein the permeation enhancer comprises a fatty chain having 8 to 14 carbon atoms.

16. The pharmaceutical composition of claim 1, wherein the permeation enhancer is selected from capric acid and salts, esters, or derivatives thereof.

17. The pharmaceutical composition of claim 16, wherein the permeation enhancer is sodium caprate or a derivative thereof.

18. The pharmaceutical composition of claim 17, wherein the content of sodium caprate or a derivative thereof ranges between about 25-300 mg, about 50-200 mg, or about 100-200 mg.

19. The pharmaceutical composition of claim 1, which is configured to deliver the therapeutic agent and the permeation enhancer to a mucosal surface.

20. The pharmaceutical composition of claim 1, which is configured to deliver the therapeutic agent to a subject in need thereof via the oral route.

21. The pharmaceutical composition of claim 1, wherein the therapeutic agent comprises a biologically active macromolecule.

22. The pharmaceutical composition of claim 21, wherein the biologically active macromolecule is selected from the group consisting of a protein, a peptide, a polysaccharide, a nucleic acid, a lipid, and a carbohydrate, and a combination thereof.

23. The pharmaceutical composition of claim 21, wherein the biologically active macromolecule is selected from the group consisting of an insulin, an erythropoietin, an interferon, a growth hormone, an exenatide, a GLP-1 agonist, a PTH, a calcitonin, a leuprolide, an octreotide, a low molecular weight heparin, and functional analogs, mutants, salts, and derivatives thereof.

24. The pharmaceutical composition of claim 23, wherein the GLP-1 agonist is an exendin or an exendin peptide analog.

25. The pharmaceutical composition of claim 24, wherein the exendin is exendin-4.

26. The pharmaceutical composition of claim 24, wherein the exendin peptide analog is selected from exenatide and salts and functional derivatives thereof.

27. The pharmaceutical composition of claim 1, wherein the composition is formulated in the form of a capsule, a tablet, a pellet, a powder, or a granule.

28. The pharmaceutical composition of claim 27, wherein the solid dosage form is produced using a direct compression process.

29. The pharmaceutical composition of claim 27, wherein the solid dosage form is produced using a non-solvent granulation process.

30. The pharmaceutical composition of claim 29, wherein the non-solvent granulation process uses a non-solvent medium selected from ethanol, isopropanol, butanol, acetone, and ethyl acetate.

31. The pharmaceutical composition of claim 27, wherein the therapeutic agent is substantially stable during storage at room temperature.

32. A method of making a pharmaceutical composition, said method comprising:
a) fabricating a solid dosage form comprising an effective amount of a therapeutic agent, a permeation enhancer and a pharmaceutically acceptable excipient; and
b) coating the solid dosage form with a bioadhesive layer comprising a bioadhesive polymer.

33. The method of claim 32, further comprising:
c) coating the solid dosage form with an impermeable or semi-permeable layer comprising an opening capable of directing a substantially unidirectional release of the therapeutic agent and the permeation enhancer from the solid dosage form.

34. The method of claim 32, wherein the opening is formed on one side of the solid dosage form using a laser ablation process.

35. The method of claim 33, wherein the order of steps b) and c) is reversed.

36. The method of claim 32, further comprising a step of coating the composition with an enteric layer.

37. A method of treating a subject in need of a therapeutic treatment, comprising administering to said subject the pharmaceutical composition of claim 1.
